(19) United States
(12) Patent Application Publication
(10) Pub. No.: US 2013/0101611 A1
(43) Pub. Date: Apr. 25, 2013

(54) CITRULLINATION OF HUMAN PEPTIDYLARGININE DEIMINASE 4 (PAD-4) REGULATES ITS FUNCTION AND IMMUNOGENICITY

(71) Applicants: Felipe Andrade, Timonium, MD (US); Antony Rosen, Pikesville, MD (US)

(72) Inventors: Felipe Andrade, Timonium, MD (US); Antony Rosen, Pikesville, MD (US)

(73) Assignee: THE JOHNS HOPKINS UNIVERSITY, Baltimore, MD (US)

(21) Appl. No.: 13/512,051

(22) PCT Filed: Nov. 24, 2010
(86) PCT No.: PCT/US10/57983

§ 371 Date: Jan. 10, 2013

Related U.S. Application Data
(60) Provisional application No. 61/264,374, filed on Nov. 25, 2009.

(51) Int. Cl. C12N 9/96; A61K 38/50; A61K 45/06; G01N 33/68

(52) U.S. Cl. CPC C12N 9/96; G01N 33/6854; A61K 38/50; A61K 45/06
USPC 424/185.1; 435/188; 435/7.1; 435/7.92

(57) ABSTRACT

The present invention provides peptidylarginine deiminase 4 (PAD-4) polypeptides comprising one or more citrullinated Arginine sites. In one embodiment, a PAD-4 polypeptide comprises a citrulline residue at the following sites: Arg-205, Arg-212, Arg-218, Arg-372, Arg-374, Arg-383, Arg-394, Arg-495, Arg-536, and Arg-544. The present invention also provides methods for detecting the presence of autoantibodies to citrullinated PAD-4. The methods of the present invention may also be used to qualify Rheumatoid Arthritis status in a subject. In other embodiments, the present invention provides methods for inducing tolerance to citrullinated PAD-4.

### STATEMENT OF GOVERNMENTAL INTEREST

This invention was made with U.S. government support under grant no. P30-AR-053503 and grant no. R37-DE-12354. The U.S. government has certain rights in the invention.

### CROSS-REFERENCE TO RELATED APPLICATIONS

This application claims the benefit of U.S. Provisional Application No. 61/264,374, filed Nov. 25, 2009; which is incorporated herein by reference in its entirety.

### REFERENCE TO SEQUENCE LISTING SUBMITTED ELECTRONICALLY

The official copy of the Sequence Listing is submitted concurrently with the specification as an ASCII formatted text file via EFS-Web, with a file name of P10941-02_Sequence Listing_ST25.txt, creation date of Nov. 24, 2010, and with a file size of 28.6 KB. The Sequence Listing filed via EFS-Web is part of the specification and is herein incorporated by reference in its entirety.

### FIELD OF THE INVENTION

The present invention relates to the field of autoimmune diseases.

### BACKGROUND OF THE INVENTION

Posttranslational modifications of proteins greatly diversify the functional repertoire of these molecules, rapidly shaping cell functions to accommodate changes in the extracellular environment. These covalent modifications produce important effects on the structure, function, and likely the immunogenicity of the target protein (1-4). Although the discovery of nonribosomally encoded citrulline in proteins was first reported _{—}50 years ago (5,6), the importance of citrullination remained unclear until the last 10 years, when 2 major discoveries brought attention to this modification. The first finding was that patients with rheumatoid arthritis (RA) produce autoantibodies that recognize epitopes containing peptidylcitrulline, and that these autoantibodies are both highly specific for diagnosis and predictive of disease severity (7,8). The second discovery was that histones become citrullinated (9), raising the possibility that, like other posttranslational histone modifications (i.e., phosphorylation, acetylation, and methylation), histone citrullination may regulate chromatin-templated nuclear events, including transcription (10,11). The functional role of histone citrullination remains unclear (12).

The peptidyl arginine deiminase (PAD) enzymes hydrolyze guanidinium side chains in peptidyl arginine to yield peptidylcitrulline and ammonia, and belong to a larger group of guanidino-modifying enzymes called the amidinotransferase superfamily (13). To date, 5 human PAD isoenzymes have been identified (14). For historical reasons, these enzymes are designated PAD-1-PAD-4 and PAD-6 (14). PAD-4 is a homodimer that is distinguished by the insertion of a nuclear localization sequence and is the only PAD localized to the cell nucleus (15,16). Among the PAD enzymes, PAD-4 has gained special attention as a potential candidate that may drive citrullination of self antigens in RA (8). The specific immune response to citrullinated proteins, the presence of increased levels of citrullinated proteins in synovial tissue and fluid from RA patients (17-19), and the genetic association of PADI4 polymorphisms with RA in some populations (20-23) strongly suggest that pathways which promote and/or restrain protein citrullination may be altered in this disease. Understanding the mechanisms that regulate PAD activity under physiologic or pathologic conditions is therefore a high priority.

### SUMMARY OF THE INVENTION

The present invention is based, in part, on the discovery that autocitrullination of PAD-4 inactivates its function and that the efficiency of this process (i.e., citrullination-induced inactivation of PAD-4) is distinct in the different PAD-4 variants relevant to RA. Multiple citrullination sites were discovered in PAD-4, and further defined Arg-372 and -374 as the potential autocitrullination targets that inactivate the enzyme. Autocitrullination also modified the structure of PAD-4, augmenting its recognition by human autoantibodies. Taken together, these findings suggest that the extent of citrullination during cell activation represents an integrated function regulated by PAD-4 activation and by the efficiency of autocitrullination-induced inactivation of PAD-4, and that this process is influenced by PAD-4 polymorphisms associated with RA. In addition, PAD-4 autocitrullination is a potential mechanism to explain its targeting by RA autoantibodies. Autocitrullination, which influences PAD-4, both enzymatically and immunologically, may play an important role in RA pathogenesis.

Accordingly, in one aspect, the present invention provides peptidylarginine deiminase 4 (PAD-4) polypeptides comprising one or more citrullinated Arginine sites. In certain embodiments, the one or more citrullinated Arginine sites are selected from the group consisting of Arg-8, Arg-123, Arg-131, Arg-137, Arg-156 Arg-205, Arg-212, Arg-218, Arg-292, Arg-372, Arg-374, Arg-383, Arg-394, Arg-419, Arg-427, Arg-441, Arg-484, Arg-488, Arg-495, Arg-536, and Arg-544, Arg-550, Arg-555, Arg-609, Arg-639, Arg-650, and Arg-651.

In other embodiments, the one or more citrullinated Arginine sites of the PAD-4 polypeptides are selected from the group consisting of Arg-205, Arg-212, Arg-218, Arg-372, Arg-374, Arg-383, Arg-394, Arg-495, Arg-536, and Arg-544. In a specific embodiment, the citrullinated Arginine sites comprise Arg-205, Arg-212, Arg-218, Arg-372, Arg-374, Arg-383, Arg-394, Arg-495, Arg-536, and Arg-544. In another specific embodiment, the citrullinated Arginine sites comprise Arg-372 and Arg-374.

In another embodiment, the present invention provides a PAD-4 polypeptide comprising a citrulline residue at the following sites: Arg-205, Arg-212, Arg-218, Arg-372, Arg-374, Arg-383, Arg-394, Arg-495, Arg-536, and Arg-544. In yet another embodiment, the present invention provides a PAD-4 polypeptide comprising a citrulline residue at the following sites: Arg-372 and Arg-374. The present invention further provides kits comprising at least one cit-PAD-4 polypeptide.

A cit-PAD-4 polypeptide may further comprise a sequence selected from the group consisting of (the underlined “R” represents a citrullinated residue):

(SEQ ID NO: 6)
VMGPDFGYVTR,
(SEQ ID NO: 7)
LLLASPRSCYK,
(SEQ ID NO: 8)
RVMGPDFGYVTR;
(SEQ ID NO: 9)
TLREHNSFVER;
(SEQ ID NO: 10)
DFFTNHTLVLHVARSEMDK;
(SEQ ID NO: 11)
RVMGPDFGYVTR;
(SEQ ID NO: 12)
TLPWFDSPRNRGLK;
(SEQ ID NO: 13)
VFQATRGK;
and
(SEQ ID NO: 14)
VRVFQATRGK.

In another aspect, the present invention relates to methods for detecting the presence of autoantibodies to citrullinated PAD-4 (cit-PAD-4 autoantibodies) in a subject. In particular embodiment, the method comprises contacting a biological sample taken from a subject with a polypeptide of the present invention and detecting the binding of the polypeptide with an autoantibody specific for the polypeptide, wherein the detection of binding is indicative of the presence of cit-PAD-4 autoantibodies in the subject. In such methods, the binding can be detected by enzyme-linked immunosorbent assay (ELISA), immunoprecipitation or immunoblotting.

In yet another aspect, the present invention relates to methods for assessing efficacy of an RA treatment regimen in a subject. In particular embodiments, the methods comprise establishing a baseline level of cit-PAD-4 autoantibodies in a subject prior to an RA treatment regimen; monitoring the level of cit-PAD-4 autoantibodies using a polypeptide of the present invention at least at one point after initiation of the RA treatment regimen; and comparing the observed level of cit-PAD-4 autoantibodies to the baseline level of cit-PAD-4 autoantibodies, wherein a decrease in the level of PAD autoantibodies is indicative of the efficacy of the RA treatment regimen. In such methods, the binding can be detected by enzyme-linked immunosorbent assay (ELISA), immunoprecipitation or immunoblotting.

In a further aspect, the present invention relates to methods for qualifying RA status in a subject. Qualifying RA status can be qualifying the risk of RA, the development of RA, the presence or absence of RA, the stage of RA, the subtype of RA, the prognosis for the subject, and the effectiveness of treatment of RA. In certain embodiments, the methods comprise measuring the level of cit-PAD-4 autoantibodies in a biological sample from the subject; and correlating the measurement with RA status. In one embodiment, the level of cit-PAD-4 autoantibodies is measured using a polypeptide described herein. In another embodiment, the level of cit-PAD-4 autoantibodies is measured by ELISA, immunoprecipitation or immunoblotting.

In one aspect, the present invention provides methods of inducing tolerance to citrullinated PAD-4 in a subject. In particular embodiments, the methods comprise administering to the subject a pharmaceutical composition comprise a cit-PAD-4 polypeptide in an amount effective to induce tolerance to citrullinated PAD-4 in the subject. The method can further comprise administering an immunosuppressive agent.

The present invention also provides vaccines comprising a cit-PAD-4 polypeptide. In certain embodiments, the vaccine elicits an immune response that suppresses or eliminates cit-PAD-4 specific autoreactive T-cells in an individual.

### BRIEF DESCRIPTION OF THE FIGURES

FIG. 1 shows the results of autocitrullination of recombinant human peptidyl arginine deiminase type 4 (rhPAD-4) in vitro. In FIGS. 1A-1E, recombinant human PAD-4 (700 nM in 1A, 1B, and 1E or 0-700 nM in 1C and 1D) was coincubated with 700 nM human recombinant histone H3.1 or incubated alone (1E) in the presence of 10 mM CaCl₂ at 37° C. for 0-60 minutes (1A and 1B), for 60 minutes (1C and 1D), or for 0-120 minutes (1E). In FIG. 1F, recombinant human PAD-4 (700 nM) was incubated in the presence of 5 mM EDTA or 10 mM CaCl₂ for 120 minutes at 37° C. After termination of the reactions, the samples were electrophoresed and noncitrullinated and citrullinated proteins were detected by immunoblotting with anti-histone H3, anti-PAD-4 C-terminal antibody, and anti-modified citrulline (AMC). In FIGS. 1G and 1H, immunoprecipitation (IP) of PAD-4 from control and activated neutrophils is shown. Primary human neutrophils in Hanks' balanced salt solution containing 2 mM CaCl₂ were incubated in the absence (lane 1) or presence (lane 2) of 1 μM ionomycin for 2 hours at 37° C. Following the incubation, the cells were lysed in radioimmunoprecipitation assay buffer. The samples were directly electrophoresed (1G and top panel of 1H) or used to immunoprecipitate endogenous PAD-4 using a rabbit anti-human PAD-4 C-terminal antibody (bottom panel of 1H). Protein citrullination was detected by anti-modified citrulline immunoblotting (1G), and endogenous PAD-4 (top panel of 1H) or immunoprecipitated PAD-4 (bottom panel of 1H) was detected by immunoblotting using the rabbit anti-PAD-4 C-terminal antibody.

FIG. 2 shows experimental results relating to the autocitrullination of PAD-4 during cell activation and the preferential recognition of citrullinated PAD-4 by human RA autoantibodies. FIG. 2A shows immunoprecipitation of control and citrullinated rhPAD-4 by rabbit antibodies and by human rheumatoid arthritis (RA) sera. Noncitrullinated (native) rhPAD-4 and citrullinated (cit) rhPAD-4 were immunoprecipitated using rabbit anti-human PAD-4 antibodies against the C-terminal (Cterm) or N-terminal (Nterm) domains, or using human anti-PAD-4 RA sera (i.e., 2454, 1067, 2489, and 2314). Purified immune complexes were electrophoresed, and PAD-4 was detected by immunoblotting using anti-PAD-4 C-terminal antibody. B, Immunoprecipitation of PAD-4 from control and activated neutrophil lysates. Cell lysates were generated in radioimmunoprecipitation assay (RIPA) buffer from control neutrophils (lanes 1 and 3) and ionomycin-activated neutrophils (lanes 2 and 4), and endogenous PAD-4 was immunoprecipitated using human anti-PAD-4 serum. Then, control and ionomycin-activated samples were divided in two and immunoblotted using anti-PAD-4 C-terminal antibody (lanes 1 and 2) or anti-modified citrulline (lanes 3 and 4). FIG. 2C shows protein citrullination in 293T-cells that were transiently transfected to express green fluorescent protein (GFP)-PAD-4. After 48 hours, the cells were incubated in the absence (lane 1) or presence (lane 2) of 1 μM ionomycin for 2 hours at 37° C. Following the incubation, the cells were lysed in RIPA buffer and electrophoresed to detect protein citrullination by anti-modified citrulline. FIG. 2D shows immunoprecipitation of GFP-PAD-4 from control and activated 293T-transfected cells. Purified immune complexes were electrophoresed, and equal protein loading was visualized by ponceau S staining (top) prior to anti-modified citrulline immunoblotting (bottom).

FIG. 3 demonstrates that autocitrullination of PAD-4 inhibits its function. FIG. 3A shows the generation of noncitrullinated rhPAD-4 (lane 1) and citrullinated rhPAD-4 (lane 2). Recombinant human PAD-4 was incubated in the absence or presence of 10 mM CaCl₂ for 120 minutes at 37° C. Samples were immunoblotted with rabbit anti-PAD-4 (top) and anti-modified citrulline (bottom). FIG. 3B reports citrullination activity of control and citrullinated rhPAD-4. HL-60 cell lysates were incubated with buffer alone (lanes 1 and 4), noncitrullinated rhPAD-4 (lanes 2 and 5), or citrullinated (cit) rhPAD-4 (lanes 3 and 6) for 15 minutes (lanes 1-3) or 60 minutes (lanes 4-6) at 37° C. After termination of the reactions, the samples were electrophoresed and immunoblotted using anti-modified citrulline, anti-PAD-4 C-terminal antibody, and anti-β-actin (loading control).

FIG. 4 shows the clustering of the potential citrullination sites in peptidyl arginine deiminase type 4 (PAD-4) into 3 distinct regions. FIG. 4A is a schematic representation of the secondary structure of PAD-4 (24). The secondary structure elements in the N-terminal and C-terminal domains are shown in blue and yellow, respectively. Bars show α-helices; arrows show β strands; and broken lines show disordered regions. The potential arginine targets for citrullination are shown in red. FIG. 4B presents the tertiary structure of PAD-4, revealing clustering of citrullination sites into 3 distinct regions. The cluster comprising Arg-372, -374, -383, and -394 spans the active site cleft. The N-terminal and C-terminal domains are shown in blue and yellow, respectively, while the sites of arginine deimination are indicated in red. The model shown was generated from the Molecular Modeling Database (National Center for Biotechnology Information), according to coordinates generated by Arita et al. (16). Adapted by permission from Macmillan Publishers Ltd, 11(8) NAT. STRUCT. MOL. BIOL. 777-83 (2004).

FIG. 5 demonstrates that the PAD-4 citrullination sites Arg-372 and Arg-374 regulate PAD-4 activity during cell activation. FIG. 5A is a schematic diagram of the interactions between Arg-374 and Arg-372 at the active site cleft of PAD-4 and N-terminal residues in histone H3-1 (26). Arg-374 makes multiple hydrogen bonds with backbone carbonyl oxygens of the Ala-7 (A7) and Arg-8 (R8) residues in histone H3.1 (green), and Arg-372 recognizes the carbonyl oxygen of the Arg-8 residue by means of water (Wat)-mediated hydrogen bonds. FIG. 5B shows the conversion of Arg-372 and Arg-374 to citrullines (Cit) during PAD-4 autocitrullination, potentially distressing the interactions shown in 5A. As presented in FIGS. 5C and 5D, the PAD-4 mutants PAD-4R372K and PAD-4R374K lack citrullination activity. The 293T-cells were mock transfected (lane 1) or transiently transfected to express PAD-4 (lane 2), PAD-4R372K (lane 3), or PAD-4R374K (lane 4). At 48 hours post-transfection, the cells were stimulated with 1 μM ionomycin for 1 hour. After termination of the reactions, the samples were electrophoresed, and equal protein loading was visualized by ponceau S staining (C) prior to anti-modified citrulline immunoblotting (bottom panel of 5D). PAD-4 expression was visualized by immunoblotting (top panel of 5D).

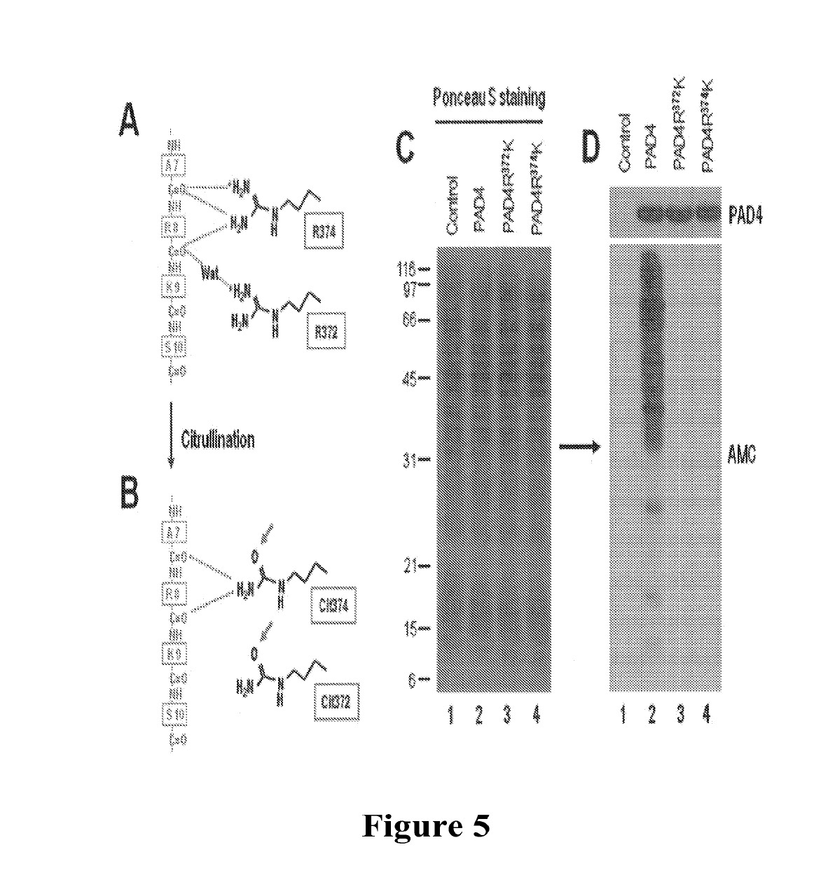

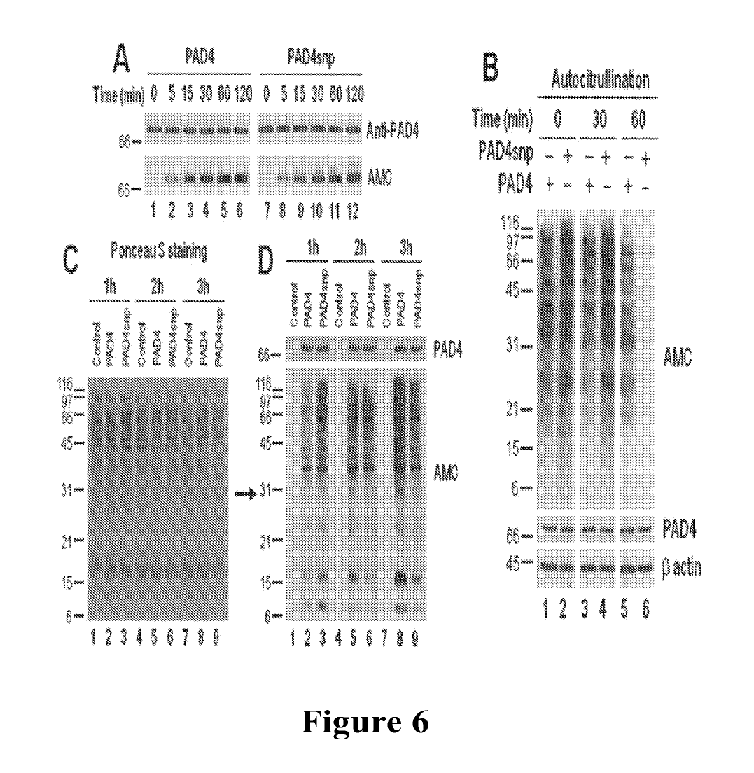

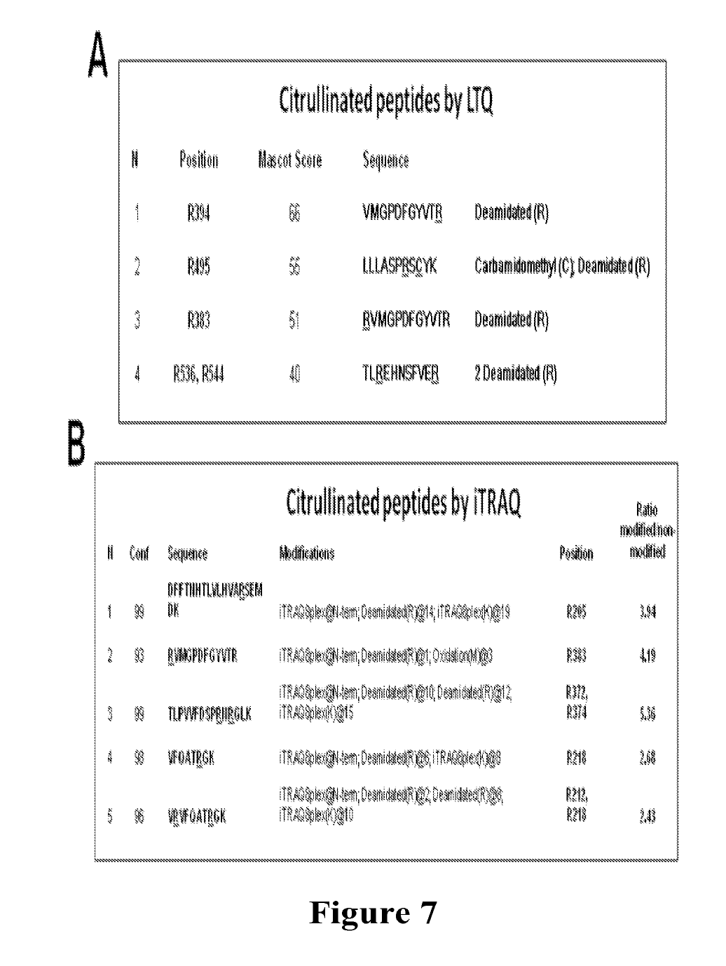

As shown in FIG. 6, PAD-4 variants have distinct patterns of inactivation induced by autocitrullination. FIG. 6A presents results of autocitrullination of rhPAD-4 and rhPAD-4-snp variants. Recombinant human PAD-4 and rhPAD-4-snp were incubated in the presence of 10 mM CaCl₂ at 37° C. for 0-120 minutes. Noncitrullinated and citrullinated proteins were detected by immunoblotting with anti-PAD-4 (top) and anti-modified citrulline (bottom), respectively. FIG. 5B shows citrullination activity of control and citrullinated rhPAD-4 and rhPAD-4-snp. Recombinant human PAD-4 (lanes 1, 3, and 5) or rhPAD-4-snp (lanes 2, 4, and 6) were incubated in the presence of 10 mM CaCl₂ for 0, 30, or 60 minutes at 37° C., and further incubated with HL-60 cell lysate for an additional 15 minutes at 37° C. After termination of the reactions, the samples were electrophoresed and immunoblotted using anti-modified citrulline, anti-PAD-4, and anti-β-actin (loading control). FIGS. 6C and 6D reports citrullination activity of PAD-4 and PAD-4-snp during cell activation. The 293T-cells were mock transfected (lanes 1, 4, and 7) or transiently transfected to express PAD-4 (lanes 2, 5, and 8) or PAD-4-snp (lanes 3, 6, and 9). At 48 hours post-transfection, the cells were stimulated with 1 μM ionomycin for 1 hour (lanes 1-3), 2 hours (lanes 4-6), 3 hours (lanes 7-9), and 5-6 hours (results not shown). After termination of the reactions, the samples were electrophoresed, and equal protein loading was visualized by ponceau S staining (C) prior to anti-modified citrulline immunoblotting (bottom panel of 6D). PAD-4 expression was visualized by immunoblotting (top panel of 6D).

FIG. 7 displays the potential citrullination sites in PAD-4. FIG. 7A shows the peptides containing citrullines identified by LTQ. The raw files were searched with Mascot using deamidation (R) as a variable modification. Mascot scores for the identified peptides are >40. FIG. 7B lists peptides containing citrullines identified by iTRAQ. Protein Pilot confidence scores for the identified peptides are >90%.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to an “autoantibody” is a reference to one or more autoantibodies, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

The following definitions are used throughout this specification. Other definitions are embedded within the specification for case of reference. As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies.

As used herein, the term “autoantibodies” refers to antibodies that are capable of reacting against an antigenic constituent of an individual's own tissue or cells (e.g., the antibodies recognize and bind to “self” antigens). In certain embodiments, the term “PAD-4 autoantibodies” refers to antibodies produced by an individual that are immunospecific to the individual's own PAD-4 protein. In other embodiments, the term “citrullinated PAD-4 autoantibodies” or “cit-PAD-4 autoantibodies” refers to antibodies produced by an individual that are immunospecific to the individual's own citrullinated PAD-4 protein.

The terms “specifically binds to,” “specific for,” and related grammatical variants refer to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

As used herein, the term “antigen” is generally used in reference to any substance that is capable of reacting with an antibody. It is intended that this term encompass any antigen and “immunogen” (i.e., a substance which induces the formation of antibodies). Thus, in an immunogenic reaction, antibodies are produced in response to the presence of an antigen (immunogen) or portion of an antigen. More specifically, the terms are used herein to describe an antigen that elicits a humoral and/or cellular immune response (i.e., is immunogenic), such that administration of the immunogen to an animal (e.g., via a vaccine of the present invention) mounts an antigen-specific immune response against the same or similar antigens that are encountered within the tissues of the animal. In another embodiment, when it is desirable to suppress an immune response against a given antigen, an antigen may comprise a toleragen (defined below).

The terms “biological sample,” “sample,” “patient sample” and the like, encompass a variety of sample types obtained from an individual and can be used in a diagnostic or monitoring assay. The definition encompasses blood and other liquid samples of biological origin (including, but not limited to, serum, plasma, urine, saliva, stool and synovial fluid), solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as CD4⁻ T lymphocytes, glial cells, macrophages, tumor cells, peripheral blood mononuclear cells (PBMC), and the like. The terms further encompass a clinical sample, and also include cells in culture, cell supernatants, tissue samples, organs, bone marrow, and the like.

The term “correlates” indicates that a phenomenon (e.g. signal intensity) is related to another phenomenon (e.g., antibody concentration, or disease severity). The relationship is typically a parallel relationship (e.g. as one increases, the other increases).

As used herein, the term “rheumatoid arthritis” refers to any disorder involving inflammation of the joints. The term refers to such features as joint erosion, lymphocyte infiltration, synovial hyperplasia, aggressive proliferation of fibroblast-like synoviocytes and macrophages.

As used herein, a “subject” or “patient” means an individual and can include domesticated animals, (e.g., cats, dogs, etc.); livestock (e.g., cattle, horses, pigs, sheep, goats, etc.), laboratory animals (e.g., mouse, rabbit, rat, guinea pig, etc.) and birds. In one aspect, the subject is a mammal such as a primate or a human. In particular, the terms refer to mammals diagnosed with a disease associated with the presence of citrullinated PAD-4 autoantibodies. More specifically, the terms refer to mammals diagnosed with rheumatoid arthritis.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

### Citrullinated Pad-4 Polypeptides

The present invention provides citrullinated PAD-4 polypeptides. As used herein, the terms “polypeptide,” “peptide,” and “protein” are used interchangeably herein to refer to polymers of amino acids of any length. Thus, peptides, oligopeptides, and proteins are included within the definition of polypeptide. This term also encompasses post expression modifications of the polypeptide, for example, glycosylations, acetylations, phosphorylations and the like. Included within the definition are, for example, polypeptides containing one or more analogues of an amino acid (including, for example, unnatural amino acids, etc.), polypeptides with substituted linkages, as well as other modifications known in the art, both naturally occurring and non-naturally occurring.

In a particular aspect, the citrullinated PAD-4 polypeptide sequences are human amino acid sequences. The present invention also encompasses both the full length PAD-4 amino acid sequences having at least one of the arginine amino acids converted to a citrulline, or fragments thereof. The limitation being that any fragment of any desired length has at least one citrulline that specifically binds to cit-PAD-4 autoantibodies. Indeed, the present invention encompasses any PAD-4 peptide that is demonstrated to be a potential target of cit-PAD-4 auto antibodies in RA patients. The full length nucleotide sequence for PAD-4 is shown at SEQ ID NO:1. The full length amino acid sequence for human PAD-4 is shown at SEQ ID NO:2.

In a particular aspect, the citrullinated PAD-4 peptides of the invention bind with high affinity to cit-PAD-4 autoantibodies. It is understood by one of skill in the art, that “high affinity” is used synonymously with the terms “specifically binds to” and “specific for” and refers to the capability of the citrullinated PAD-4 peptides to bind with higher or increased affinity to a cit-PAD-4 autoantibody as compared with a non-citrullinated PAD-4 antibody. It is further understood that such binding affinity can be readily established for example in vitro using a peptide binding assay in which a sample peptide is used to displace a standard peptide.

Accordingly, in a specific embodiment, the present invention provides a PAD-4 polypeptide comprising one or more citrullinated Arginine sites. In another embodiment, the one or more citrullinated Arginine sites are selected from the group consisting of Arg-8, Arg-123, Arg-131, Arg-137, Arg-156 Arg-205, Arg-212, Arg-218, Arg-292, Arg-372, Arg-374, Arg-383, Arg-394, Arg-419, Arg-427, Arg-441, Arg-484, Arg-488, Arg-495, Arg-536, and Arg-544, Arg-550, Arg-555, Arg-609, Arg-639, Arg-650, and Arg-651 (SEQ ID NO:3). In a more specific embodiment, the one or more citrullinated Arginine sites are selected from the group consisting of Arg-205, Arg-212, Arg-218, Arg-372, Arg-374, Arg-383, Arg-394, Arg-495, Arg-536, and Arg-544.

In a further embodiment, the citrullinated Arginine sites comprise Arg-205, Arg-212, Arg-218, Arg-372, Arg-374, Arg-383, Arg-394, Arg-495, Arg-536, and Arg-544 (SEQ ID NO:4). In yet another embodiment, the citrullinated Arginine sites comprise Arg-372 and Arg-374 (SEQ ID NO:5).

In another specific embodiment, the present invention provides a PAD-4 polypeptide comprising a citrulline residue at the following sites: Arg-205, Arg-212, Arg-218, Arg-372, Arg-374, Arg-383, Arg-394, Arg-495, Arg-536, and Arg-544. In yet another embodiment, the present invention provides a PAD-4 polypeptide comprising a citrulline residue at the following sites: Arg-372 and Arg-374.

A cit-PAD-4 polypeptide may further comprise a sequence selected from the group consisting of (the underlined “R” represents a citrullinated residue): VMGPDFGYVTR (SEQ ID NO:6), LLLASPRSCYK (SEQ ID NO:7), RVMGPDFGYVTR (SEQ ID NO:8); TLREHNSFVER (SEQ ID NO:9); DFFTNHTLVLHVARSEMDK (SEQ ID NO:10); RVMGPDFGYVTR (SEQ ID NO:11); TLPWFDSPRNRGLK (SEQ ID NO:12); VFQATRGK (SEQ ID NO:13); and VRVFQATRGK (SEQ ID NO:14). Methods for generating peptides are well known in the art. Therefore, additional citrullinated PAD-4 polypeptides are within the scope of the invention. The replacement of one or more arginine residues with a citrulline residue to generate a multitude of citrullinated PAD-4 polypeptides (of any length) and subsequent analyses thereof can be carried out without undue experimentation.

Certain of these polypeptide sequences may contain additional arginines that may be converted to citrulline. Generally, the PAD-4 polypeptides of the present invention may comprise any suitable length for specific recognition by cit-PAD-4 autoantibodies. In one embodiment, the citrullinated PAD-4 polypeptide is a full length protein. In other embodiments, the citrullinated PAD-4 polypeptide may comprise at least about 2 amino acids to about 663 amino acids in length. More specifically, the citrullinated PAD-4 polypeptides may comprise at least about 8 or 9 amino acids to several hundred amino acids in length. Even more specifically, the citrullinated PAD polypeptides may comprise at least about 10-20 amino acids in length to at least about 100 amino acids in length. Furthermore the citrullinated PAD polypeptides of the present invention may comprise about 9 to about 50 amino acids in length and include any ranges of length therein (i.e., 9-50, 9-45, 9-40, 9-35, 9-30, 9-25, 9-20, 9-15, etc.) as is understood by one of skill in the art. Peptides of over about 50 amino acids in length are also encompassed by the present invention. The length of polypeptide being only restricted by its binding capability to specifically bind cit-PAD-4 autoantibodies.

The polypeptides of the present invention may also include multipeptides. In the context of the present invention, a multipeptide is a molecule comprised of at least two antigenic peptide units, i.e. combinations of peptide units that may or may not be linked by a covalent bond. Such multipeptides may be comprised of linear, branched, cyclic peptide units or a combination of these. Multipeptides may be comprised both of peptide units having the same amino acid sequence, and of peptide units having different amino acid sequences. A multipeptide according to the invention comprises at least 7, preferably at least 10 amino acids, i.e. the peptide units may overlap. In specific embodiments, the present invention may comprise cyclic versions of citrullinated PAD-4 polypeptides.

In addition, the citrullinated PAD-4 polypeptides of the present invention may also include dimers and trimers of the peptides as well as additional stabilizing flanking sequences as is understood by those of skill in the art and described for example in U.S. Pat. No. 6,184,204 and U.S. Pat. No. 5,824,315. A multimer according to the invention can either be a homomer, consisting of a multitude of the same peptide, or a heteromer consisting of different peptides. As stated, the amino acid sequences of the polypeptides according to the invention can be flanked by random amino acid sequences. Preferred are flanking sequences that have a stabilizing effect on the polypeptides, thus increasing their biological availability. In addition, other peptidomimetics are also useful in the polypeptides of the present invention. For a general review, see A. F. Spatola, in CHEMISTRY AND BIOCHEMISTRY OF AMINO ACIDS, PEPTIDES AND PROTEINS, B. Weinstein, eds., Marcel Dekker, New York, p. 267 (1983). The polypeptides of the invention also encompass polypeptides that have been modified by, for example, phosphorylation, glycosylation or lipidation.

Furthermore, the polypeptides of the present invention may also encompass “functionally equivalent variants” or “analogues” of the polypeptides. As such, this would include but not be limited to polypeptides with partial sequence homology, polypeptides having one or more specific conservative and/or non-conservative amino acid changes and polypeptide conjugates which do not alter the biological or structural properties of the polypeptide.

In terms of “functional analogues”, it is well understood by those skilled in the art, that inherent in the definition of a biologically functional polypeptide analogue is the concept that there is a limit to the number of changes that may be made within a defined portion of the molecule and still result in a molecule with an acceptable level of equivalent biological activity. A plurality of distinct polypeptides with different substitutions may easily be made and used in accordance with the invention. It is also understood that certain residues are particularly important to the biological or structural properties of a polypeptide, and such residues may not generally be exchanged.

Functional analogues can be generated by conservative or non-conservative amino acid substitutions. Amino acid substitutions are generally based on the relative similarity of the amino acid side-chain substituents, for example, their hydrophobicity, hydrophilicity, charge, size and the like. Thus, within the scope of the invention, conservative amino acid changes means, an amino acid change at a particular position which is of the same type as originally present; i.e. a hydrophobic amino acid exchanged for a hydrophobic amino acid, a basic amino acid for a basic amino acid, etc. Examples of conservative substitutions include the substitution of non-polar (hydrophobic) residues such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another, the substitution of a branched chain amino acid, such as isoleucine, leucine, or valine for another, the substitution of one aromatic amino acid, such as phenylalanine, tyrosine or tryptophan for another. Such amino acid changes result in functional analogues in that they do not significantly alter the overall charge and/or configuration of the polypeptide. Examples of such conservative changes are well-known to the skilled artisan and are within the scope of the present invention. Conservative substitution also includes the use of a chemically derivatized residue in place of a non-derivatized residue provided that the resulting polypeptide is a biologically functional equivalent to the polypeptides of the invention. Therefore, the citrullinated polypeptides of the present invention encompass a polypeptide having an amino acid sequence that differs from SEQ ID Nos. 2-5 by one or more conservative amino acid substitutions. The citrullinated polypeptides of the invention also encompass a polypeptide having an amino acid sequence that differs from SEQ ID Nos. 2-5 by a single mutation, where the single mutation represents a single amino acid deletion, insertion or substitution.

The present invention further provides citrullinated PAD-4 peptides. The citrullinated PAD-4 peptides of the present invention may be made by methods known to those of skill in the art most notably and preferably by chemical synthesis using techniques well known in the chemistry of proteins such as solid phase synthesis (Merrifield et al., 65 J. AM. CHEM. ASSOC. 2149 (1964); Merrifield et al., 85 J. AMER. CHEM. SOC. 2149 (1963); and Merrifield et al., 35 INT. J. PEPTIDE PROTEIN RES. 161-214 (1990)) or synthesis in homogenous solution (METHODS OF ORGANIC CHEMISTRY, E. Wansch (Ed.) Vol. 15, pts. I and II, Thieme, Stuttgart (1987)) to generate synthetic peptides. Citrulline is a post-translationally modified arginine that is created through the process of deimination which is catalyzed by the enzyme peptidylarginine deiminase 4 (PAD-4) that removes a positive charge from arginine and makes the resulting citrulline polar in nature.

In one embodiment, citrullinated peptides of the invention can be made from known commercially available sources of PAD-4. In this aspect, lyophilized PAD-4 is reconstituted in an appropriate buffer to which the enzyme peptidylarginine deiminase 4 is added. Alternatively, Ca²⁺ is added to PAD-4 in solution. The solution is allowed to stand at an appropriate temperature for a time sufficient to cause modification of arginine residues to citrulline and thus create a citrullinated PAD-4 protein. The citrullinated protein is then isolated by the removal of the enzyme using a high molecular weight membrane to separate the enzyme or other methods of chromatography. One of skill in the art will understand that the temperature of incubation, buffer condition and time of incubation may vary depending on the protein that is being deiminated (Masson-Bessiere et al., 166 J. IMMUNOL. 4177-4184 (2001)).

The citrullinated PAD-4 proteins may be further isolated and purified by methods selected on the basis of properties revealed by its sequence. Purification can be achieved by protein purification procedures such as chromatography methods (gel-filtration, ion-exchange and immunoaffinity), by high-performance liquid chromatography (HPLC, RP-HPLC, ion-exchange HPLC, size-exclusion HPLC, high-performance chromatofocusing and hydrophobic interaction chromatography) or by precipitation (immunoprecipitation). Polyacrylamide gel electrophoresis can also be used to isolate the citrullinated proteins based on the molecular weight of the protein, charge properties and hydrophobicity. The purified citrullinated proteins can be used in further biochemical analyses to establish secondary and tertiary structure which may aid in the design of pharmaceuticals to interact with the protein, alter the protein charge configuration or charge interaction with other proteins or alter its function.

### III. Assays of Citrullinated PAD-4 Autoantibodies; Kits

The present invention provides compositions and methods for using citrullinated PAD-4 polypeptides. In several embodiments, the citrullinated PAD-4 polypeptides can be used to assay for the presence of cit-PAD-4 autoantibodies. In a specific embodiment, a method for detecting the presence of autoantibodies to citrullinated PAD-4 in a subject comprises contacting a biological sample taken from a subject with a citrullinated PAD-4 polypeptide, and detecting the binding of the polypeptide with an autoantibody specific for the polypeptide, wherein the detection of binding is indicative of the presence of citrullinated PAD-4 autoantibodies in the subject.

Methods for assaying such autoantibodies are described herein in and known to those of ordinary skill in the art. For example, an immunoassay can be used to detect and analyze autoantibodies in a biological sample. As used herein, the term “immunoassay” is used in reference to any method in which antibodies are used in the detection of an antigen. It is contemplated that a range of immunoassay formats be encompassed by this definition, including but not limited to, direct immunoassays, indirect immunoassays, and “sandwich immunoassays.” However, it is not intended that the present invention be limited to any particular format. It is contemplated that other formats, including radioimmunoassays (RIA), immunofluorescent assays (IFA), and other assay formats, including, but not limited to, variations on the ELISA, RIA and/or IFA methods will be useful in the methods of the present invention. The term also includes immunoprecipitation and immunoblotting.

Thus, in one aspect, the methods of the present invention include using a sandwich assay to detect the cit-PAD-4 autoantibodies. Sandwich assays generally involve the use of two binding agents, e.g., antibodies, each capable of binding to a different portion, or epitope, of the protein(s) to be detected and/or quantitated. In a sandwich assay, the analyte is typically bound by a first binding agent which is immobilized on a solid support, and thereafter a second binding agent binds to the analyte, thus forming an insoluble complex. See, e.g., U.S. Pat. No. 4,376,110. Alternatively, the sandwich assay may be performed in solution, also referred to as a homogeneous assay. See, e.g., U.S. Pat. No. 7,413,862.

In some embodiments of these methods, a capture probe including a first binding agent is capable of specifically binding to a disease-associated antigen, e.g., a PAD-4 polypeptide, which is bound to one or more autoantibodies. In turn, the detection probe including a second binding agent binds to the autoantibodies. Thus, in this particular example, a four-part complex is formed between: (1) the capture probe, (2) the disease-associated antigen, (3) the autoantibody, and (4) the detection probe. In an alternative embodiment, the positions of the first and second binding agents are reversed, such that the capture probe attached to the solid support is capable of specifically binding to the autoantibodies and the detection probe is capable of specifically binding to the disease-associated antigen.

As stated above, the methods can be performed using any immunological technique known to those skilled in the art of immunochemistry. As examples, ELISA, immunofluorescence, radioimmunoassays or similar techniques may be utilized. In general, an appropriate capture probe is immobilized on a solid surface and the sample to be tested (e.g., human serum) is brought into contact with the capture probe. For example, modified glass substrates that covalently or non-covalently bind proteins can be used to bind the disease-associated antigen. The substrate may be treated with suitable blocking agents to minimize non-specific binding. If the autoantibody is present in the sample, a complex between the autoantibody and the capture probe is formed. A detection probe is then added, which specifically recognizes an epitope of a human immunoglobulin (Ig), if present. The anti-human immunoglobulin detection probe may be directed against the Fc region of the human antibody and with as little cross-reactivity as possible against the capture antibody species.

In another embodiment, the methods comprise contacting a sample with a capture probe including an antibody capable of binding to a disease-associated antigen. The sample is also contacted with a detection probe including anti-human Ig antibodies. The presence, absence, and/or amount of the complex may be detected, wherein the presence or absence of the complex is indicative of the presence or absence of the autoantibodies.

The complex can then be detected or quantitatively measured using methods well-known in the art. The detection probe may be labeled with biochemical markers such as, for example, a nanoparticle, horseradish peroxidase (HRP) or alkaline phosphatase (AP), and detection of the complex can be achieved by the addition of a substrate for the enzyme which generates a calorimetric, chemiluminescent or fluorescent product. Alternatively, the presence of the complex may be determined by addition of a marker protein labeled with a detectable label, for example an appropriate enzyme. In this case, the amount of enzymatic activity measured is inversely proportional to the quantity of complex formed and a negative control is needed as a reference to determine the presence of antigen in the sample. Another method for detecting the complex may utilize antibodies or antigens that have been labeled with radioisotopes followed by measure of radioactivity.

The sample may be contacted with the detection probe before, after, or simultaneously with the capture probe. In one embodiment, the sample is first contacted with the detection probe so that autoantibodies present in the sample bind to the detection probe to form a target analyte complex. The mixture is then contacted with the substrate having capture probes bound thereto so that the target analyte complex binds to the capture probe on the substrate. In another embodiment, the sample is first contacted with the substrate so that a target analyte complex present in the sample binds to a capture probe, and the target analyte complex bound to the capture probe is then contacted with the detection probe so that the autoantibodies bind to the detection probe. In another embodiment, the sample, the detection probe and the capture probe on the substrate are contacted simultaneously.

The present invention further provides kits for commercial sale. In certain embodiments, the kit may comprise at least one cit-PAD-4 polypeptide. The kit may comprise the equipment, solutions and/or instructions necessary for all steps in the process of creating cit-PAD-4 polypeptides, and the like.

### IV. Methods for Qualifying Disease Status

The present invention can also be used to qualify disease status in a subject. In one aspect, the compositions and methods of the present invention may be used to qualify autoimmune disease status in a subject. In a more specific aspect, the compositions and methods of the present invention may be used to qualify Rheumatoid Arthritis status in a subject. The term “Rheumatoid Arthritis status” or “RA status” refers to the status of the disease in the patient or subject. Examples of types of RA statuses include, but are not limited to, the subject's risk of RA, the development of RA, the presence or absence of RA, the stage of RA in a subject, the subtype of RA, the prognosis for the subject, and the effectiveness of treatment of the disease. Other statuses and degrees of each status are known in the art.

Thus, in a specific embodiment, a method for qualifying RA status in a subject comprises measuring the level of cit-PAD-4 autoantibodies in a biological sample from the subject; and correlating the measurement with RA status. The level of cit-PAD-4 autoantibodies may be measured using a polypeptide described herein. More specifically, the level of cit-PAD-4 autoantibodies may be measured by immunoassay including, but not limited to, ELISA, immunoprecipitation or immunoblotting.

In a further aspect, the methods and compositions of the present invention are useful in the status qualification and/or treatment of any disease marked by the presence of autoantibodies to citrullinated PAD 4. Thus, the citrullinated PAD-4 polypeptides of the present invention find utility in qualifying the status of and/or treating arthritis and other autoimmune diseases. “Autoimmune” disorders include any disorder, condition, or disease in which the immune system mounts a reaction against self cells or tissues, due to a breakdown in the ability to distinguish self from non-self or otherwise. Examples of autoimmune disorders include, but are not limited to, Hashimoto's thyroiditis, pernicious anemia, Addison's disease, type I diabetes, rheumatoid arthritis, systemic lupus erythematosus, dermatomyositis, Sjogren's syndrome, lupus erythematosus, multiple sclerosis, myasthenia gravis, Reiter's syndrome, Grave's disease, polymyositis, Guillain Barre, Wegener's granulomatosus, polyarteritis nodosa, polymyalgia rheumatica, temporal arteritis, Bechet's disease, Churg-Strauss syndrome, Takayasu's arteritis, and the like. Autoimmune disorders can involve any component of the immune system, and can target any cell or tissue type in the body.

Disease status, e.g., Rheumatoid Arthritis status, may be correlated in relation to a reference level. As used herein, the term “reference level” is intended to mean a control level of a biomarker, e.g., a disease-associated autoantibody, used to evaluate a test level of the biomarker in a biological sample from an individual. A reference level can be a normal reference level or a disease-state reference level. A normal reference level is an amount of a biomarker in a non-diseased subject or subjects. A disease-state reference level is an amount of a biomarker in a subject with a positive diagnosis for the disease or condition. A reference level also can be a stage-specific reference level. A stage-specific reference level refers to a level of a biomarker characteristic of a given stage of progression of a disease or condition.

The reference level used for comparison with the measured or tested level for an autoantibody may vary, depending on the aspect of the invention being practiced, as will be understood from the foregoing discussion. For disease diagnostic methods, the “reference level” is typically a predetermined reference level, such as an average of levels obtained from a population that is not afflicted with the disease or medical condition, but in some instances, the reference level can be a mean or median level from a group of individuals including diseased patients. In some instances, the predetermined reference level is derived from (e.g., is the mean or median of) levels obtained from an age-matched population. Alternatively, the reference level may be a historical reference level for the particular patient (e.g., a disease-associated antigen or autoantibody level that was obtained from a sample derived from the same individual, but at an earlier point in time).

For disease staging or stratification methods (i.e., methods of classifying diseased patients into mild, moderate and severe stages of disease), the reference level is normally a predetermined reference level that is the mean or median of levels from a population which has been diagnosed with disease. In some instances, the predetermined reference level is derived from (e.g., is the mean or median of) levels obtained from an age-matched population.

Age-matched populations (from which reference values may be obtained) are ideally the same age as the individual being tested, but approximately age-matched populations are also acceptable. Approximately age-matched populations may be within 1, 2, 3, 4, or 5 years of the age of the individual tested, or may be groups of different ages which encompass the age of the individual being tested.

Approximately age-matched populations may be in 2, 3, 4, 5, 6, 7, 8, 9, or 10 year increments (e.g. a “5 year increment” group which serves as the source for reference values for a 62 year old individual might include 58-62 year old individuals, 59-63 year old individuals, 60-64 year old individuals, 61-65 year old individuals, or 62-66 year old individuals).

The process of comparing a measured value and a reference value can be carried out in any convenient manner appropriate to the type of measured value and reference value for the autoantibody at issue. Measuring can be performed using quantitative or qualitative measurement techniques, and the mode of comparing a measured value and a reference value can vary depending on the measurement technology employed. For example, when a qualitative assay is used to measure autoantibody levels, the levels may be compared by comparing data from densitometric or spectrometric measurements (e.g., comparing numerical data or graphical data, such as bar charts, derived from the measuring device). However, it is expected that the measured values used in the methods of the invention will most commonly be quantitative values (e.g., quantitative measurements of signal intensity).

A measured value is generally considered to be substantially equal to or greater than a reference value if it is at least 95% of the value of the reference value (e.g., a measured value of 1.71 would be considered substantially equal to a reference value of 1.80). A measured value is considered less than a reference value if the measured value is less than 95% of the reference value (e.g., a measured value of 1.7 would be considered less than a reference value of 1.80). A measured value is considered more than a reference value if the measured value is at least more than 5% greater than the reference value (e.g., a measured value of 1.89 would be considered more than a reference value of 1.80).

The process of comparing may be manual (such as visual inspection by the practitioner of the method) or it may be automated. For example, an assay device may include circuitry and software enabling it to compare a measured value with a reference value for an autoantibody. Alternatively, a separate device (e.g., a digital computer) may be used to compare the measured value(s) and the reference value(s). Automated devices for comparison may include stored reference values for the autoantibody being measured, or they may compare the measured value(s) with reference values that are derived from contemporaneously measured reference samples.

In some embodiments, the methods of the present invention utilize “simple” or “binary” comparison between the measured level(s) and the reference level(s) (e.g., the comparison between a measured level and a reference level determines whether the measured level is higher or lower than the reference level). For autoantibody levels, a comparison showing that the measured value for the autoantibody is higher than the reference value indicates or suggests a diagnosis of disease.

In certain aspects, the comparison is performed to determine the magnitude of the difference between the measured and reference values (e.g., comparing the “fold” or percentage difference between the measured value and the reference value). A fold difference that is about equal to or greater than the minimum fold difference disclosed herein suggests or indicates a diagnosis of a disease or medical condition, as appropriate to the particular method being practiced. A fold difference can be determined by measuring the absolute concentration of the disease-associated antigen or autoantibody and comparing that to the absolute value of a reference, or a fold difference can be measured by the relative difference between a reference value and a sample value, where neither value is a measure of absolute concentration, and/or where both values are measured simultaneously.

As will be apparent to those of skill in the art, when replicate measurements are taken for the biomarker(s) tested, the measured value that is compared with the reference value is a value that takes into account the replicate measurements. The replicate measurements may be taken into account by using either the mean or median of the measured values as the “measured value.”

### IV. Pharmaceutical Compositions Comprising a Citrullinated PAD-4 Polypeptide

The present invention further provides pharmaceutical compositions. In one aspect, the cit-PAD-4 polypeptides of the present invention are useful as a toleragen. In a specific embodiment, the cit-PAD-4 polypeptides may be useful as a toleragen in active and established RA. In another embodiment, cit-PAD-4 polypeptides may be used to absorb cit-PAD-4 autoantibodies from RA patients. In yet another embodiment, cit-PAD-4 polypeptides may be used to vaccinate subjects at risk for developing RA.

In one aspect, the cit-PAD-4 polypeptides of the present invention may be useful as a toleragen. In one embodiment, the cit-PAD-4 polypeptides can be used as a toleragen in individuals predisposed to or afflicted with a disease or condition characterized by the presence of cit-PAD-4 autoantibodies. In a specific embodiment, the cit-PAD-4 polypeptides are useful as a toleragen in subjects predisposed to or afflicted with an autoimmune disease. In a more specific embodiment, the cit-PAD-4 polypeptides are useful as a toleragen in subjects predisposed to or afflicted with RA.

As used herein, the term “toleragen” means any antigen (such as a polypeptide, polynucleotide, carbohydrate, lipid, or combination of any thereof) that mediates host unresponsiveness. By way of example, a toleragen works by inducing the tolerized host not to produce antibodies or cell-mediated immune responses specific for the toleragen. Additional discussion of toleragens may be found, for instance, in PCT publication WO 2006/052668, which is incorporated herein in its entirety. More specifically, a “toleragen” refers to an antigen that is provided in a form, amount, or route of administration such that there is a reduced or changed immune response to the antigen, and in particular embodiments, substantial non-responsiveness, anergy, other inactivation, or deletion of immune system cells in response to contact with the toleragen or a cell expressing or presenting such toleragen.

With respect to the use of cit-PAD-4 polypeptides as toleragens, the term “inducing tolerance” or “inducing immune tolerance” means the prevention, reduction and/or stabilization of the extent of an immune response to an immunogen. An “immune response” may be humoral and/or cellular, and may be measured using standard assays known in the art. For purposes of this invention, the immune response is generally reflected by the presence of cit-PAD-4 autoantibodies. Quantitatively the reduction (as measured by reduction in auto antibody production) is at least about 5%, at least about 10%, at least about 25%, at least about 50%, at least about 75%, at least about 90%, at least about 95%, or at least about 99%. It is understood that the tolerance is antigen-specific, and applies for purposes of the invention to those individuals having cit-PAD-4 autoantibodies. “Inducing tolerance” also includes slowing and/or delaying the rate of increase of autoantibody level.

Accordingly, in particular embodiments, a method of inducing tolerance to citrullinated PAD-4 in a subject comprises administering to the subject a pharmaceutical composition comprise a cit-PAD-4 polypeptide in an amount effective to induce tolerance to citrullinated PAD-4 in the subject. In other embodiments, the method can further comprises administering an immunosuppressive agent. Indeed, antigen specific immune tolerance can be induced in a subject by administration of a toleragen in combination with a regimen of immunosuppression. In this regard, the methods may optionally include a preceding conditioning period, where immunosuppressive agents are administered in the absence of the toleragen. After the tolerizing regimen, the subject is withdrawn from the suppressive agents, but is able to maintain specific immune tolerance to the immunogenic epitopes present on the toleragen. Maintenance doses of the toleragen are optionally administered after the tolerizing regimen is completed. In particular embodiments, the toleragen will have high uptake properties that allow uptake in vivo at low concentrations in a wide variety of tolerizing cell types.

In particular aspects of the present invention, it should be noted that the dose of the immunosuppressive agent is to be sufficient to substantially suppress T-cells. Variations in dosage of the drugs may be combined to reach the same degree of T-cell suppression in different subjects and under different conditions. The level of T-cell suppression is monitored as that level at which the T-cells do not proliferate in response to antigen stimulation. Methods for monitoring T-cell proliferation are known to those skill in the art, and may be used in conjunction with the present invention.

As used herein, T-cell immunosuppressive agents are compounds that inhibit the activity of T-cells, particularly T-helper cells, usually without general suppression of the proliferation and activity of other cells, such as B cells, monocytes, bone marrow hematopoietic progenitors cells, etc. Methods of assaying for T-cell immunosuppression are well known in the art, include in vitro assays such as release of IL-2 by T-helper cells in the presence of antigen, incorporation of ³H thymidine into DNA in the presence of antigen or a stimulant such as Con A, release of ⁵¹Cr in the presence of allogeneic stimulatory cells, etc. In vivo assays may rely upon measuring the proliferation of T-cells, the release of cytokines, inability to reject a graft while actively suppressed, and the like.

A group of compounds of particular interest for these purposes are the immunophilins, also referred to as calcineurin inhibitors, which inhibit T-helper cells. Calcineurin is a Ca²⁺/calmodulin-dependent S/T protein phosphatase 2B, which has been reported to be important in the calcium signaling pathway. This enzyme is a heterodimer of a 61 kDa calmodulin-binding catalytic subunit (calcineurin A) and a small (19 kDa) regulatory subunit (calcineurin B). The immunosuppressive drugs, cyclosporin A, rapamycin, FK506, etc inhibit calcineurin, which is necessary for the nuclear import of NF-AT (nuclear factor of activated T-cells). Importantly, the dose of T-cell immunosuppressive agent for the purpose of tolerization may be higher than that normally used for general immunosuppression.

Immunosuppressive agents may also comprise antiproliferative agents. Antiproliferative agents, for the purposes of the methods of the present invention, are pharmaceutically active compounds that depress cellular proliferation. As the cells of the immune system are often actively dividing, even general anti-proliferative agents frequently have an immunosuppressive effect. Many such anti-proliferative drugs are known in the art, for example as used in chemotherapy.

Accordingly, anti-proliferative agents of interest may include antimetabolites, e.g., nucleotide analogs such as azathioprine, 6-mercaptopurine, thioguanine, cytarabine, etc.; other analogs, such as methotrexate, mycophenolic acid, or 6-(1,3-Dihydro-4-hydroxy-6-methoxy-7-methyl-3-oxy-5-isobenzofuranyl)-4-methyl-4-hexanoic acid, and the like. Alkylating agents such as cyclophosphamide, chlorambucil, etc., may also find use as immunosuppressive antiproliferatives, for example where there is a pre-existing immune response to the antigen of interest. In specific embodiments of the present invention, the antiproliferative agent is azathioprine (AZA) or 6-mercaptopurine (6-MP).

In another aspect, the cit-PAD-4 polypeptides of the present invention may be useful as a vaccine. In one embodiment, the cit-PAD-4 polypeptides can be used as a vaccine in individuals predisposed to or otherwise at risk for a disease or condition characterized by the presence of cit-PAD-4 autoantibodies. In a specific embodiment, the cit-PAD-4 polypeptides are useful as a vaccine in subjects predisposed to or otherwise at risk for an autoimmune disease. In a more specific embodiment, the cit-PAD-4 polypeptides are useful as a vaccine in subjects predisposed to or otherwise at risk for RA.

Thus, in yet another embodiment of the invention relates to a vaccine comprising a cit-PAD-4 polypeptide, wherein the vaccine elicits an immune response that suppresses or eliminates cit-PAD-4 polypeptides autoreactive T-cells in an individual. As used here, a “vaccine or a “vaccinating antigen” can be an immunogen or a toleragen, but is an antigen used in a vaccine, where a biological response (elicitation of an immune response, tolerance) is to be elicited against the vaccinating antigen. A vaccine is a specific type of composition that is used to immunize or tolerize an animal against a particular antigen. Accordingly, a vaccine comprises at least one compound or agent that elicits an immune response against an antigen or immunogenic or toleragenic portion thereof, as a result of administration of the vaccine. Administration of a vaccine preferably results in a protective or therapeutic effect, wherein subsequent exposure to the antigen (or a source of the antigen) elicits an immune response against the antigen (or source) that reduces or prevents a disease or condition in the animal. Such immune responses can generally enhance or suppress the immune response to the antigen and in the case of cit-PAD-4 polypeptides, it is preferred that a vaccine suppress an immune response against cit-PAD-4 polypeptides. The concept of vaccination is well known in the art. The immune response that is elicited by administration of a pharmaceutical composition of the present invention can be any detectable change in any facet of the immune response (e.g., cellular response, humoral response, cytokine production), as compared to in the absence of the administration of the vaccine.

Accordingly, a pharmaceutical composition of the present invention may comprise an effective amount of at least one of the cit-PAD-4 polypeptides described herein. As used herein, the term “effective,” means adequate to accomplish a desired, expected, or intended result. More particularly, an “effective amount” or a “therapeutically effective amount” is used interchangeably and refers to an amount of a cit-PAD-4 polypeptide of the present invention, either alone or in combination with another therapeutic agent, necessary to provide the desired therapeutic effect, e.g., an amount that is effective to prevent, alleviate, treat or ameliorate symptoms of disease or prolong the survival of the subject being treated. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

The pharmaceutical compositions of the present invention are in biologically compatible form suitable for administration in vivo for subjects. The pharmaceutical compositions further comprise a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the cit-PAD-4 polypeptide is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition may also contain minor amounts of wetting or emulsifying agents, or pH buffering agents.

The pharmaceutical compositions of the present invention can take the form of solutions, suspensions, emulsions, tablets, pills, capsules, powders, sustained-release formulations and the like. The composition can be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. In a specific embodiment, a pharmaceutical composition comprises an effective amount of a cit-PAD-4 polypeptide together with a suitable amount of a pharmaceutically acceptable carrier so as to provide the form for proper administration to the patient. The formulation should suit the mode of administration.

The pharmaceutical compositions of the present invention may be administered by any particular route of administration including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means. Most suitable routes are oral administration or injection.

In general, the pharmaceutical compositions disclosed herein may be used alone or in concert with other therapeutic agents at appropriate dosages defined by routine testing in order to obtain optimal efficacy while minimizing any potential toxicity. The dosage regimen utilizing a pharmaceutical composition of the present invention may be selected in accordance with a variety of factors including type, species, age, weight, sex, medical condition of the patient; the severity of the condition to be treated; the route of administration; the renal and hepatic function of the patient; and the particular pharmaceutical composition employed. A physician of ordinary skill can readily determine and prescribe the effective amount of the pharmaceutical composition (and potentially other agents including therapeutic agents) required to prevent, counter, or arrest the progress of the condition.

Optimal precision in achieving concentrations of the therapeutic regimen (e.g., a pharmaceutical composition comprising a cit-PAD-4 polypeptide in combination with another therapeutic agent) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical composition and various therapeutic agents may be independently optimized and combined to achieve a synergistic result wherein the pathology is reduced more than it would be if either was used alone.

In particular, toxicity and therapeutic efficacy of a pharmaceutical composition disclosed herein may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ratio LD₅₀/ED₅₀. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED₅₀ with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC₅₀ (the concentration of the test composition that achieves a half-maximal inhibition of symptoms) as determined in cell culture. Such information may be used to accurately determine useful doses in humans. Levels in plasma may be measured, for example, by high performance liquid chromatography.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See WO 00/67776, which is entirely expressly incorporated herein by reference.

More specifically, the pharmaceutical compositions may be administered in a single daily dose, or the total daily dosage may be administered in divided doses of two, three, or four times daily. In the case of oral administration, the daily dosage of the compositions may be varied over a wide range from about 0.1 ng to about 1,000 mg per patient, per day. The range may more particularly be from about 0.001 ng/kg to 10 mg/kg of body weight per day, about 0.1-100 μg, about 1.0-50 μg or about 1.0-20 mg per day for adults (at about 60 kg).

The daily dosage of the pharmaceutical compositions may be varied over a wide range from about 0.1 ng to about 1000 mg per adult human per day. For oral administration, the compositions may be provided in the form of tablets containing from about 0.1 ng to about 1000 mg of the composition or 0.1, 0.2, 0.5, 1.0, 2.0, 5.0, 10.0, 15.0, 100, 150, 200, 250, 300, 350, 400, 450, 500, 550, 600, 650, 700, 800, 900, or 1000 milligrams of the composition for the symptomatic adjustment of the dosage to the patient to be treated. An effective amount of the pharmaceutical composition is ordinarily supplied at a dosage level of from about 0.1 ng/kg to about 20 mg/kg of body weight per day. In one embodiment, the range is from about 0.2 ng/kg to about 10 mg/kg of body weight per day. In another embodiment, the range is from about 0.5 ng/kg to about 10 mg/kg of body weight per day. The pharmaceutical compositions may be administered on a regimen of about 1 to about 10 times per day.

In the case of injections, it is usually convenient to give by an intravenous route in an amount of about 0.0001 μg-30 mg, about 0.01 μg-20 mg or about 0.01-10 mg per day to adults (at about 60 kg). In the case of other animals, the dose calculated for 60 kg may be administered as well.

Doses of a pharmaceutical composition of the present invention can optionally include 0.0001 μg to 1,000 mg/kg/administration, or 0.001 μg to 100.0 mg/kg/administration, from 0.01 μg to 10 mg/kg/administration, from 0.1 μg to 10 mg/kg/administration, including, but not limited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration or any range, value or fraction thereof.

As a non-limiting example, treatment of subjects can be provided as a one-time or periodic dosage of a composition of the present invention 0.1 ng to 100 mg/kg such as 0.0001, 0.001, 0.01, 0.1 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Specifically, the pharmaceutical compositions of the present invention may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

More specifically, the pharmaceutical compositions may be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

Alternatively, the pharmaceutical compositions may be administered about once every day, about once every 2 days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days.

The pharmaceutical compositions of the present invention may alternatively be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks.

Alternatively, the pharmaceutical compositions of the present invention may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

Alternatively, the pharmaceutical compositions may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

Alternatively the pharmaceutical compositions may be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

It would be readily apparent to one of ordinary skill in the art that the pharmaceutical compositions of the present invention (e.g., the cit-PAD-4 polypeptides) can be combined with one or more therapeutic agents. In particular, the compositions of the present invention and other therapeutic agents can be administered simultaneously or sequentially by the same or different routes of administration. The determination of the identity and amount of therapeutic agent(s) for use in the methods of the present invention can be readily made by ordinarily skilled medical practitioners using standard techniques known in the art. In specific embodiments, the cit-PAD-4 polypeptides of the present invention can be administered in combination with an effective amount of a therapeutic agent that treats RA and/or any disease associated with the presence of cit-PAD-4 autoantibodies.

In another aspect, the cit-PAD-4 polypeptides of the present invention may be combined with other therapeutic agents including, but not limited to, immunomodulatory agents, anti-inflammatory agents (e.g., adrenocorticoids, corticosteroids (e.g., beclomethasone, budesonide, flunisolide, fluticasone, triamcinolone, methylprednisolone, prednisolone, prednisone, hydrocortisone), glucocorticoids, steroids, and non-steroidal anti-inflammatory drugs (e.g., aspirin, ibuprofen, diclofenac, and COX-2 inhibitors).

In various embodiments, the cit-PAD-4 polypeptides of the present invention in combination with a second therapeutic agent may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In particular embodiments, two or more therapies are administered within the same patient visit.

In certain embodiments, the cit-PAD-4 polypeptides of the present invention and one or more other therapies arc cyclically administered. Cycling therapy involves the administration of a first therapy (e.g., the cit-PAD-4 polypeptides) for a period of time, followed by the administration of a second therapy (e.g. another therapeutic agent) for a period of time, optionally, followed by the administration of a third therapy for a period of time and so forth, and repeating this sequential administration, e.g., the cycle, in order to reduce the development of resistance to one of the therapies, to avoid or reduce the side effects of one of the therapies, and/or to improve the efficacy of the therapies. In certain embodiments, the administration of the combination therapy of the present invention may be repeated and the administrations may be separated by at least 1 day, 2 days, 3 days, 5 days, 10 days, 15 days, 30 days, 45 days, 2 months, 75 days, 3 months, or at least 6 months.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Human PAD-4 Cloning, Expression Vectors, and Recombinant Human PAD-4 (rhPAD-4) Purification.

Total RNA was purified from ATRA-differentiated HL-60 cells and reverse transcribed to generate complementary DNA (cDNA). PAD-4 cDNA was amplified by polymerase chain reaction and cloned into the Gateway (Invitrogen) vector pDEST-51 for mammalian expression and the pDEST-17 prokaryotic expression vector to generate an N-terminal His₆-tagged fusion protein that was further purified by AKTA Prime Chromatography system (Amersham Biosciences). The purified protein was dialyzed against 10 mM Tris (pH 7.4), 300 mM NaCl, 200 μM dithiothreitol, 1 mM EDTA, and 10% glycerol. The purity of the preparation was >98% as assessed by sodium dodecyl sulfate (SDS)-polyacrylamide gel electrophoresis and Coomassie blue staining (data not shown). To generate plasmids encoding PAD-4 containing 3 nonsynonymous polymorphisms associated with RA susceptibility (PAD-4-snp [Gly55→Ser, Val182→Ala, and Gly112→Ala]), PAD-4^{R372K}, PAD-4^{R374K}, and PAD-4 cDNA into pDEST-51 was used as template for site-directed mutagenesis (Stratagene). The cDNA encoding PAD-4-snp was also cloned into pDEST-17 for protein purification as described above.

Cell Culture and Cell Transfection.

HL-60 and 293T-cells were cultured using standard procedures. Cell lines were transfected using Lipofectamine 2000 (Invitrogen).

In Vitro Citrullination Assays.

Using siliconized tubes (Sigma), 700 nM human rhPAD-4 was incubated alone or 0-700 nM rhPAD-4 was coincubated with 700 nM human recombinant histone H3.1 (New England Biolabs) in buffer A (i.e., 100 mM Tris [pH 7.6]) in the absence or presence of 10 mM CaCl₂ or 5 mM EDTA as indicated in the figure legends. After 0-120 minutes at 37° C., reactions were stopped by adding SDS sample buffer and boiling. In some experiments, following rhPAD-4 incubation (i.e., after 0, 30, 60, or 120 minutes) in the absence or presence of 10 mM CaCl₂, 200 nM noncitrullinated or citrullinated rhPAD-4 was further incubated in the presence of HL-60 cell lysate (7×10⁶ cells/ml) generated in buffer B (100 mM Tris [pH 7.6], 150 mM NaCl, 1% Nonidet P40 [NP40], and protease inhibitors), plus 10 mM CaCl². After 15 or 60 minutes at 37° C., reactions were stopped by adding SDS sample buffer and boiling. Protein citrullination was determined by anti-modified citrulline immunoblotting, according to the recommendations of the manufacturer (Millipore). Additionally, noncitrullinated and citrullinated rhPAD-4 were also used for mass spectrometry or immunoprecipitation assays. For these studies, 700 nM rhPAD-4 was incubated for 2 hours, which corresponds to the time at which PAD-4 autocitrullination reaches its plateau.

Neutrophil Isolation.

After institutional review board approval and informed consent were obtained, neutrophils were isolated from heparinized venous blood from healthy volunteers. Briefly, after Ficoll-Hypaque isolation of mononuclear cells, neutrophils were isolated by 2 cycles of red blood cell lysis using ACK lysing buffer (Quality Biological). The purified neutrophils were washed and resuspended at 3×10⁶ cells/ml in Hanks' balanced salt solution without calcium/magnesium. At this point, neutrophil viability was typically >98%, as assessed by trypan blue dye exclusion.

Ca²⁺/Ionomycin-Induced Cell Activation.

Neutrophils or 293T-cells were stimulated with or without 1 μM ionomycin (Sigma) for 1-3 hours at 37° C. as indicated in the figure legends. Following the incubation, cells were lysed in radioimmunoprecipitation assay (RIPA) buffer containing protease inhibitors, sonicated, and cleared by centrifugation. The supernatants were further used for immunoprecipitation and/or immunoblotting. In some experiments, following the incubation, the cells were directly lysed and boiled in SDS sample buffer for further analysis by immunoblotting. Protein citrullination was determined by anti-modified citrulline immunoblotting.

Antibodies and Immunoprecipitation Assays.

Antihuman PAD-4 antibodies recognizing the C-terminal region (amino acids 519-528) were generated in rabbit (Covance) and affinity-purified using PAD-4 peptide 519-528. The anti-human PAD-4 N-terminal (amino acids 1-15) antibody was from Sigma, and the anti-green fluorescent protein (anti-GFP) antibody was from Invitrogen. Human anti-PAD-4 antisera have been described previously (24). Four anti-PAD-4 sera confirmed by Western blotting were randomly selected for the immunoprecipitation assays. Immunoprecipitations were performed using neutrophil or 293T-cell lysate in RIPA buffer, and noncitrullinated or citrullinated rhPAD-4 (10 μg/ml) in buffer C (20 mM Tris, 150 mM NaCl, 1 mM EDTA, 1% NP40 [pH 7.4]), as indicated in the figure legends.

Mass Spectrometry Analysis.

Native or in vitro autocitrullinated PAD-4 were reduced, alkylated, and digested with trypsin (sequencing grade; Promega). Peptides were analyzed by liquid chromatography/tandem mass spectrometry on an LTQ ion trap mass spectrometer (Thermo Fisher Scientific) or a QSTAR/Pulsar mass spectrometer (Applied Biosystems/MDX Sciex) interfaced with a 2-dimensional nanoLC system (Eksigent). Peptides were fractionated in a reverse-phase C18 (5 μm, 120 Å, YMC ODS-AQ; Waters) 75 μm×100 mm column with a 10-μm emitter using 0-60% acetonitrile/0.5% formic acid gradient over 30 minutes at 300 nl/minute. For iTRAQ experiments, the iTRAQ reagents were dissolved in isopropanol, added to the digests, and incubated at room temperature for 2 hours. After labeling, the combined peptide mixture was fractionated by strong cation exchange chromatography on an Agilent high-performance liquid chromatography system using a PolySulfoethyl A column (2.1×100 mm, 5 μm, 300 Å) (PolyLC). The absorbance at 214 nm was monitored, and 5 strong cation exchange fractions were collected along the gradient.

Peptide sequences were identified using Mascot (www.matrixscience.com) or ProteinPilot (Applied Biosystems) software to search the National Center for Biotechnology Information nonredundant database with acquired fragmentation data using human as species, trypsin as enzyme (one missed cleavage allowed), Arg deamination as a variable modification, and cysteine static modification with methylmethanethiosulfate or amines with iTRAQ 8. The peptide confidence threshold cutoff for this study was at least 95% confidence using Mascot and 90% confidence using ProteinPilot.

### Example 1

### Self-Citrullination of rhPAD-4 In Vitro

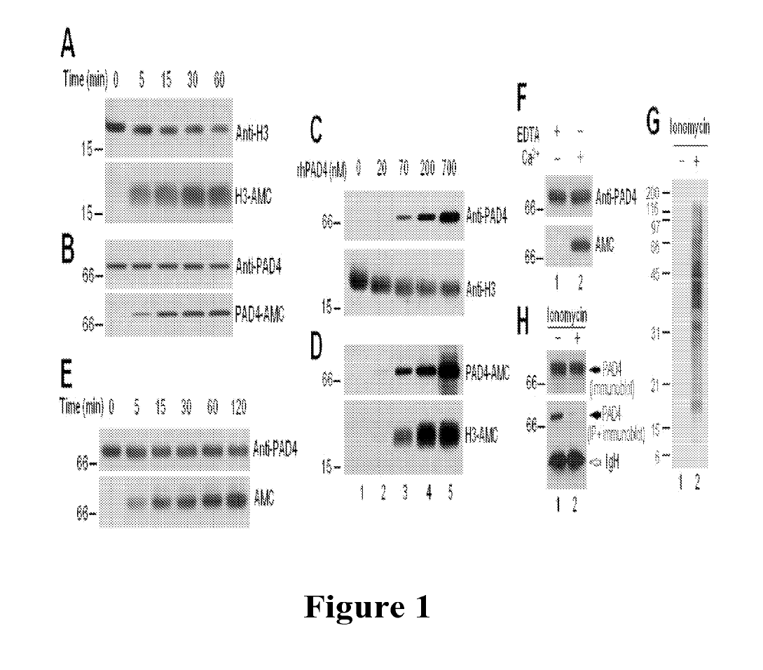

In preliminary in vitro studies of histone citrullination by PAD-4, rhPAD-4 is autocitrullinated. When recombinant histone H3.1 was coincubated with rhPAD-4, both proteins became citrullinated in a time-dependent manner, as determined by anti-modified citrulline immunoblotting (FIGS. 1A and B). Interestingly, citrullination of rhPAD-4 is as efficient as histone H3.1 citrullination even in the presence of excess amounts of substrate (FIGS. 1C and D), suggesting that citrullination of rhPAD-4 likely involves intramolecular and/or intermolecular events within the PAD-4 dimer complex. Citrullination of rhPAD-4 is therefore not affected by enzyme concentration. Furthermore, the incubation of rhPAD-4 alone in the absence of any substrate induced a similar pattern of autocitrullination of the enzyme (FIG. 1E). Autocitrullination was confirmed to be calcium dependent (FIG. 1F).

### Example 2

### Modification of the Native Structure of PAD-4 by Citrullination

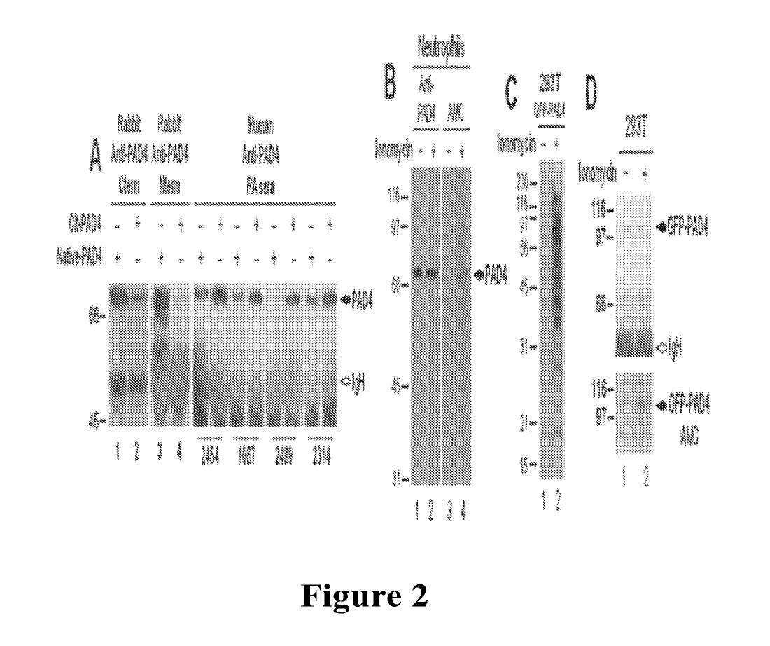

To further address whether PAD-4 is self-citrullinated in vivo, it was initially attempted to isolate endogenous PAD-4 by immunoprecipitation using a rabbit anti-human PAD-4 antibody that recognizes an epitope in the C-terminal region of PAD-4 (amino acids 519-528). Lysates were generated from control neutrophils or activated neutrophils in which citrullination was induced by Ca²⁺/ionomycin (FIG. 1G). Surprisingly, although PAD-4 was detected by immunoblotting using the anti-PAD-4 C-terminal antibody in both control and ionomycin-activated neutrophils, the antibody precipitated PAD-4 from control but not activated neutrophil lysates (FIG. 1H). Thus, although this antibody recognizes denatured PAD-4 equally in control and ionomycin-activated neutrophils, the same antibody is unable to immunoprecipitate native PAD-4 exclusively from ionomycin-activated neutrophils. Since protein citrullination produces changes in protein structure (3), it is likely that autocitrullination of PAD-4 alters its structure and recognition by antibodies generated against noncitrullinated PAD-4. To further investigate this, a similar approach was taken using PAD-4 that was citrullinated in vitro. Two different rabbit anti-PAD-4 antibodies (recognizing C-terminal epitopes [amino acids 519-528] and N-terminal epitopes [amino acids 1-15; Sigma] of human PAD-4) efficiently immunoprecipitated control but not citrullinated rhPAD-4 (FIG. 2A, lanes 1-4). Because these rabbit antibodies were generated against small peptides derived from the PAD-4 molecule, they have a very restricted recognition and, therefore, they may lose reactivity against the citrullinated form of the protein. Since RA autoantibodies preferentially recognize citrullinated autoantigens, and PAD-4 is an important antibody target in RA (24,25), it was investigated whether human anti-PAD-4 sera might recognize citrullinated PAD-4. Notably, in contrast to the rabbit anti-PAD-4 antibodies, human anti-PAD-4 RA sera immunoprecipitated both noncitrullinated and citrullinated rhPAD-4, with a general preference for the citrullinated form (FIG. 2A). In one case (serum 2489), the RA serum recognized citrullinated PAD-4 alone by immunoprecipitation. Taken together, these data demonstrate that citrullination of PAD-4 strikingly modifies the structure of the protein, decreasing recognition by antipeptide rabbit antibodies, and maintaining and enhancing its recognition by human RA autoantibodies.

### Example 3

### Citrullination of PAD-4 During Cell Activation

Using human anti-PAD-4, it was evaluated whether PAD-4 is autocitrullinated in vivo. Endogenous PAD-4 was immunoprecipitated from lysates generated from control or ionomycin-activated neutrophils using human anti-PAD-4 sera, and PAD-4 citrullination was evaluated by anti-modified citrulline immunoblotting. Notably, although RA sera immunoprecipitated PAD-4 from both control and activated neutrophil lysates (FIG. 2B, lanes 1 and 2), a prominent citrullinated protein with molecular weight identical to that of PAD-4 was detected exclusively in lysates from ionomycin-activated neutrophils (FIG. 2B, lane 4). These data strongly suggest that PAD-4 is indeed citrullinated in activated primary neutrophils.

Since RA sera may contain antibodies that could precipitate other citrullinated proteins with a molecular weight similar to that of PAD-4, a mammalian expression vector in which PAD-4 was N-terminally tagged with GFP was generated. GFP-PAD-4 was expressed by transient transfection in 293T-cells, and the GFP tag was used to immunoprecipitate PAD-4. Prominent protein citrullination was induced when GFP-PAD-4-expressing 293T-cells were incubated in the presence of Ca²⁺/ionomycin (FIG. 2C), demonstrating that GFP-PAD-4 is functional. This was similar to 293T-cells expressing the untagged PAD-4 construct (as discussed below). GFP-PAD-4 was efficiently immunoprecipitated from both control and ionomycin 293T transfected cells (FIG. 2D, top panel). However, citrullination of GFP-PAD-4 was only detected in cells activated with the calcium ionophore (FIG. 2D, bottom panel). Taken together, these data demonstrate that upon PAD-4 activation in cells, PAD-4 itself is a genuine target for citrullination.

### Example 4

### Self-Citrullination of PAD-4 Inhibits its Function

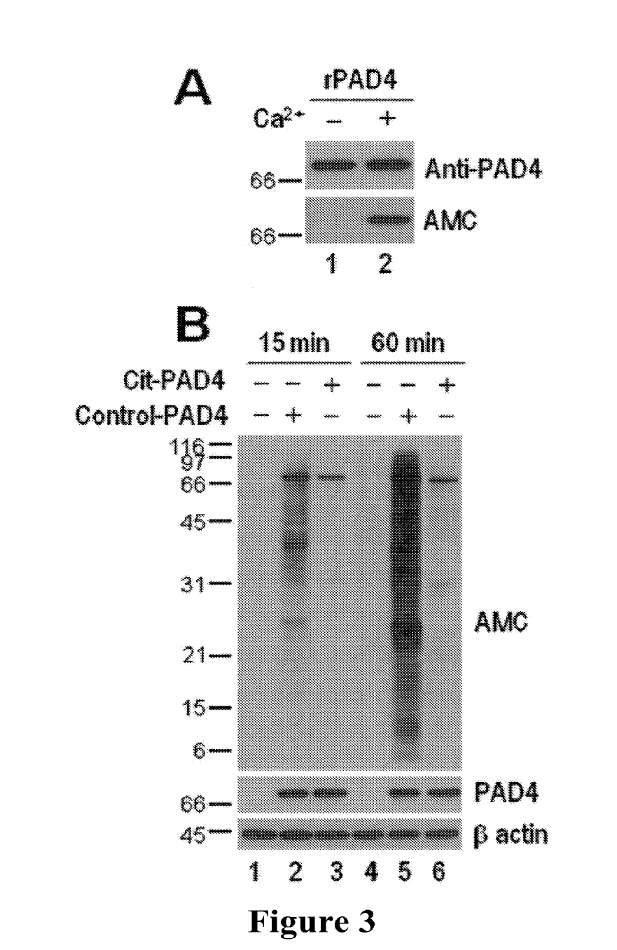

To determine the functional consequences of PAD-4 citrullination, the function of rhPAD-4 (control and citrullinated) against macromolecular substrates was directly measured. Thus, rhPAD-4 was incubated for 2 hours in the absence or presence of Ca²⁺ to generate noncitrullinated and citrullinated rhPAD-4 (FIG. 3A). These were further incubated for 15 minutes and 60 minutes in the presence of HL-60 cell lysate as a source of macromolecular targets for citrullination (FIG. 3B). As a negative control, HL-60 cell lysate was incubated alone. In the absence of added PAD-4, no citrullination was detected in the lysates by anti-modified citrulline immunoblotting at either 15 minutes or 60 minutes of incubation (FIG. 3B, top panel, lanes 1 and 4). When noncitrullinated rhPAD-4 was coincubated with the cell lysate, time-dependent protein citrullination was observed (FIG. 3B, top panel, lanes 2 and 5). In contrast, citrullinated PAD-4 had minimal citrullination activity (FIG. 3B, top panel, lanes 3 and 6). No differences were found in the levels of control and citrullinated rhPAD-4 by immunoblotting, either at 15 minutes or 60 minutes of incubation in the lysates (FIG. 3B, middle panel), confirming that these differences in PAD-4 activity did not result from rhPAD-4 degradation. These data demonstrate that autocitrullination of PAD-4 inactivates the function of the enzyme.

### Example 5

### Potential Citrullination Sites in PAD-4

Citrullination is a posttranslational modification on arginine and is manifested as a loss of 0.98 daltons as compared with the mass of the “original” arginine. Two different methods were used to identify the possible autocitrullination sites on PAD-4. The first method consisted of in-solution digestion of citrullinated PAD-4 with trypsin and subsequent analysis on an LTQ ion trap mass spectrometer. The raw data file was searched with Mascot, using Deamidation (R) as a variable modification (deamidation has the same mass change as deimination). See FIG. 7. Using this method, 5 possible sites were identified as Arg-383, Arg-394, Arg-495, Arg-536, and Arg-544.

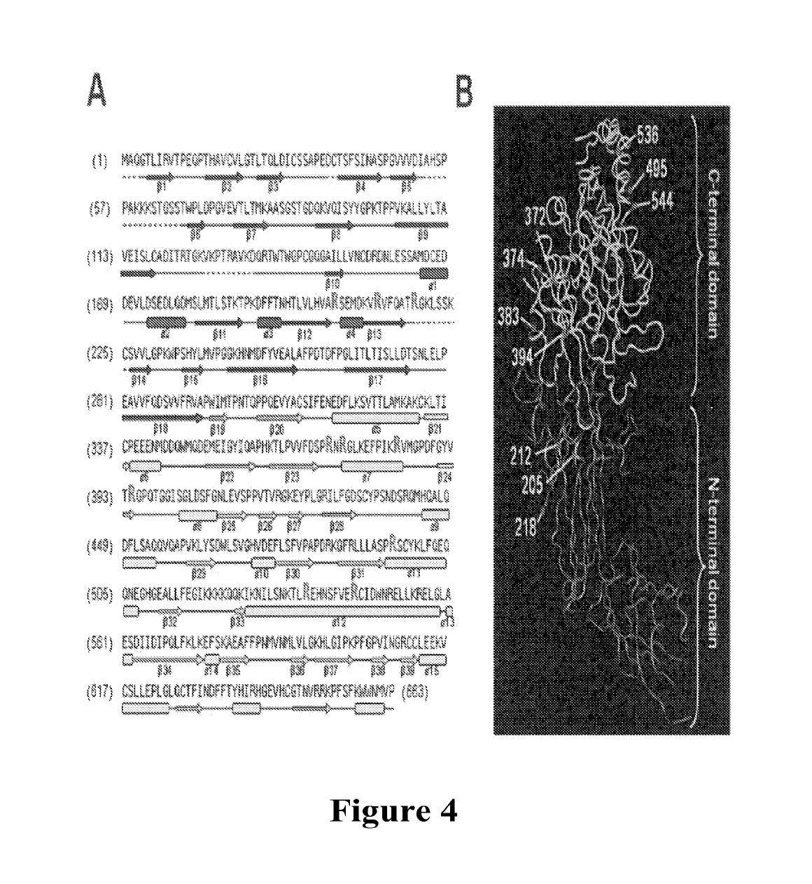

The second approach was using iTRAQ technology and analysis on a QSTAR mass spectrometer. The iTRAQ technology enables relative quantitation of possible citrullination sites as compared with the unoccupied sites. Thus, native and citrullinated PAD-4 were independently digested and labeled, mixed together, and analyzed on the QSTAR. The raw files were searched with ProteinPilot software. See FIG. 7B. The advantage of this approach is that it gives ratios between citrullinated peptide in nonmodified and modified samples. Citrullinated sites obtained by this method were Arg-205, Arg-212, Arg-218, Arg-372, Arg-374, and Arg-383. The complementary results probably arise from differences between the duty cycles and mass analyzers in the different mass spectrometers, as well as the possibility that iTRAQ labeling in the second approach may change the ionization efficiency of peptides and enable one to see peptides that might not be identified otherwise. Interestingly, the 10 citrullination sites are clustered in 3 regions within the PAD-4 molecule: one N-terminal region and 2 C-terminal regions (FIG. 4). The N-terminal cluster is located in the PAD-4 subdomain 2 (based on the crystal structure of PAD-4 [16,26]) and includes Arg-205, -212, and -218. In the C-terminal region, one cluster is located at the surface of _-helix 11 and _-helix 12 (Arg-495, -536, and -544), and the last cluster spans the active site cleft of PAD-4 (Arg-372, -374, -383, and -394). In this regard, Arg-372 and -374 are known to be directly involved in the recognition of substrate by the enzyme (26), and Arg-374 is absolutely required for citrullination of the target (26).

### Example 6

### Mechanisms of Autocitrullination-Induced Inactivation of PAD-4

To further define the functional consequences of PAD-4 autocitrullination during cell activation, those citrullination sites that surround the active site cleft in PAD-4 (26) were studied. In this regard, the study of the tertiary structure of PAD-4 in complex with histone N-terminal peptides (26) has shown that the citrullination targets Arg-372 and Arg-374 are directly involved in substrate recognition (FIG. 5A) and therefore, by affecting the enzyme-substrate interactions (FIG. 5B), their citrullination may explain the inactivation of PAD-4.

Arg-372 and Arg-374 were changed to the conserved amino acid lysine (i.e., PAD-4R372K and PAD-4R374K, respectively) to preserve charge but to lack the guanidino group removed during citrullination. Wildtype and mutant PAD-4 were expressed by transient transfection in 293T-cells, and their citrullination activity was assessed by anti-modified citrulline immunoblotting in ionomycin-activated cells. Similar PAD-4 expression was observed among the wild-type and mutant variants (FIG. 5D, top panel), and no citrullination activity was found in activated mock-transfected cells (FIG. 5D, bottom panel, lane 1). Although protein citrullination was prominent in cells expressing wild-type PAD-4 (FIG. 5D, bottom panel, lane 2), no citrullination activity was observed upon cell activation in cells expressing the PAD-4R372K and PAD-4R374K mutants (FIG. 5D, bottom panel, lanes 3 and 4), highlighting the absolute requirement of the guanidino group of arginine at those sites in PAD-4 function, and strongly indicating that the modification of this group during citrullination events around the active site cleft (FIGS. 5A and B) may be responsible for inhibition of enzyme function.

### Example 7

### Functional Consequences of Autocitrullination in Polymorphic PAD-4 Variants

To gain insight into potential differences among PAD-4 structural variants relevant to RA, studies were focused on whether the efficiency of autocitrullination and/or citrullination-induced inactivation differed between the “nonsusceptible” variant of PAD-4 (used in the experiments described above) and the PAD-4 variant that has been associated with RA (referred to as PAD-4-snp in this article) (20-23). Interestingly, although the kinetics of autocitrullination of rhPAD-4 and rhPAD-4-snp were similar (FIG. 6A), a striking difference was found in the pattern of citrullination-induced inactivation. Recombinant PAD-4 and rhPAD-4-snp underwent autocitrullination for increasing time periods (i.e., 0, 30, and 60 minutes), and their residual activity was determined by further incubation for 15 minutes in the presence of HL-60 cell lysate (as a source of citrullination targets). This time of incubation was selected after preliminary experiments showed that protein citrullination by PAD-4 falls within the linear range of the assay at this time point. Interestingly, the rhPAD-4-snp variant showed enhanced activity compared with rhPAD-4 when the enzymes were noncitrullinated (i.e., time 0), as well as early after autocitrullination (i.e., 30 minutes of autocitrullination before adding the substrate) (anti-modified citrulline in FIG. 6B). However, as the process of autocitrullination progressed (i.e., 60 minutes postcitrullination), rhPAD-4 clearly retained better enzyme activity than rhPAD-4-snp (anti-modified citrulline in FIG. 6B).

To further address whether the functional differences observed using purified PAD-4 variants also occur during cell activation, the patterns of protein citrullination during activation of 293T-cells that express PAD-4 or PAD-4-snp by transient transfection were defined. The data were fully consistent in the 2 systems. Thus, cells expressing PAD-4 showed prominent accumulation of citrullinated proteins over time, reaching a maximum at 3 hours after activation (anti-modified citrulline in FIG. 6D). This time point may represent the point at which the enzyme is largely inactivated. In contrast, although cells expressing PAD-4-snp showed a slight increase in protein citrullination compared with those expressing PAD-4 during early activation (anti-modified citrulline at 1 and 2 hours) (FIG. 6D), the PAD-4-snp cells showed decreased citrullination at later time points (anti-modified citrulline at 3 hours) (FIG. 6D). Taken together, these data suggest that PAD-4-snp is more efficiently inactivated than PAD-4.

### DISCUSSION

Protein citrullination is emerging as an important posttranslational modification in both human disease and gene regulation. Among the PAD enzymes, PAD-4 has recently become a target of significant interest because of its suspected role in the pathogenesis of RA, and also because of its potential role in gene regulation. However, significant questions about PAD-4 regulation remain unanswered. Using biochemical and cellular approaches in primary human cells and cell lines, it was demonstrated that PAD-4 is autocitrullinated in vitro and during cell activation, and that autocitrullination of PAD-4 has important effects on both the structure and the function of the enzyme. Autocitrullination thus provides an efficient mechanism of autoregulation that limits PAD-4-mediated protein citrullination.

Arginine deimination in PAD-4 occurs at 10 sites, corresponding to 37% of the arginine content in the enzyme and introducing a net of 1.5% citrullines in PAD-4. This magnitude of citrulline content can begin to induce disorder in organized protein structures (3). Indeed, striking changes in PAD-4 structure upon autocitrullination are clearly evident in the loss of citrullinated PAD-4 recognition by immunoprecipitation when using multiple different anti-PAD-4 antibodies. In contrast, human RA sera containing anti-PAD-4 antibodies recognize both noncitrullinated and citrullinated PAD-4 by immunoprecipitation, with a preference for the citrullinated form. Although it is not yet clear whether the same human antibodies recognize both noncitrullinated and citrullinated PAD-4, the recognition of citrullinated PAD-4 by RA sera is very clear, and quite distinct from the 2 rabbit anti-PAD-4 antibodies. The data suggest that these autoantibodies either recognize a part of PAD-4 whose access does not change significantly after citrullination or potentially that they recognize the modified molecule.

Based on the tertiary structure of PAD-4 in complex with histone N-terminal peptides (26), studies were focused on autocitrullination-induced inactivation of PAD-4 on those arginine residues located around the active site cleft in PAD-4, which were demonstrated to undergo citrullination. Among these sites, Arg-372 and Arg-374 are especially interesting because they are directly involved in substrate recognition (26) and, therefore, their citrullination may have critical consequences for the function of the enzyme. Indeed, the present disclosure demonstrated that the guanidino group of the arginines at positions 372 and 374 is absolutely required for efficient PAD-4-mediated citrullination, as evidenced by the lack of citrullination activity of PAD-4 mutants in which these residues were changed to lysine. These results are consistent with the observation that the guanidino groups of these arginines make critical interactions with backbone carbonyl oxygens in the histone peptides (FIG. 5A), stabilizing the substrate structure within the active site cleft of the enzyme (26).

The lysine substitution used in this study is more conservative than the alanine replacement that has been used previously (26) and provides stronger evidence of a critical role of the guanidino group, which is converted to a ureido group during citrullination. It is proposed that citrullination at these sites, by affecting substrate recognition by the enzyme, plays an essential role in autocitrullination-induced inactivation of PAD-4. However, further studies may be conducted to determine whether other citrullination sites may also affect the function of PAD-4 and whether arginines 372 and 374 are the only citrullination targets for PAD-4 inactivation. Regardless, the present data suggest that the inactivation of PAD-4 through autocitrullination may play a role in limiting the production of citrullinated proteins, ensuring that protein citrullination is appropriate to physiologic state.

Despite the genetic association of PADI4 polymorphisms with RA in some populations (20-23), the role (if any) of the PAD-4 structural variant that is associated with RA susceptibility (PAD-4-snp) remains unclear. When it was explored whether the process of citrullination-induced inactivation of PAD-4 differed among the “nonsusceptible” and the “susceptible” PAD-4 variants, it was found that while PAD-4-snp was a little more active initially, it lost function more rapidly and more completely after autocitrullination. This was a highly reproducible observation, both in assays using purified components as well as in intacT-cell systems. Since the overall kinetics of autocitrullination appear to be similar in PAD-4 and PAD-4-snp, this enhanced inhibition of PAD-4-snp may reflect more efficient deimination of “critical” activity-related arginines (i.e., 372 and/or 374) in PAD-4-snp. In support of this idea, the internal location of these arginines into the catalytic site of PAD-4 strongly supports that deimination at these sites is mediated through intramolecular events. Thus, taking into account that PAD-4-snp appears to be more active than PAD-4, the initial high rate of PAD-4-snp citrullination may also increase autocitrullination at the active site cleft and, consequently, induce a more rapid inactivation of the enzyme. To evaluate this hypothesis, further studies will require the development of novel tools to directly define the arginine-specific kinetics of autocitrullination among PAD-4 and PAD-4-snp during cell activation.

The present findings appear to be inconsistent with current models that posit that the effect of PAD-4-snp in RA is due to enhanced accumulation of citrullination in target antigens. Several possibilities are relevant in this regard. First, the activity of PAD-4-snp appears to be higher than that of the nonsusceptible variant initially, but is later obscured by more efficient inactivation. It is possible that early citrullination activity is particularly important in the pathogenesis of RA. Second, PAD-4 (specifically PAD-4-snp) may exert its effects on RA disease susceptibility as a consequence of its immunogenicity, rather than its enzyme activity. In support of this hypothesis is the observation that the production of anti-PAD-4 antibodies is strongly associated with PAD-4-snp (24), and that anti-PAD-4 antibodies are associated with more severe, erosive disease in RA (24,27,28).

Thus, it is possible that PAD-4-snp structure may change more dramatically upon autocitrullination or that autocitrullination may occur at different and/or additional sites compared with PAD-4, allowing a more efficient inactivation of the enzyme, and modifying immunogenicity. Other PADs (e.g., PAD-2) may have more critical roles in the production of citrullinated proteins targeted in RA. Regardless of the mechanism, the present findings suggest that the distinct inhibition pattern of PAD-4-snp by autocitrullination reflects an important difference, which should be further evaluated. Demonstrating the presence of citrullinated PAD-4 in vivo in RA remains an important priority.

### REFERENCES

1. Gelato K A, Fischle W. Role of histone modifications in defining chromatin structure and function. Biol Chem 2008; 389:353-63.
2. Zhang Q, Wang Y. High mobility group proteins and their post-translational modifications. Biochim Biophys Acta 2008; 1784: 1159-66.
3. Tarcsa E, Marekov L N, Mei G, Melino G, Lee S C, Steinert P M. Protein unfolding by peptidylarginine deiminase: substrate specificity and structural relationships of the natural substrates trichohyalin and filaggrin. J Biol Chem 1996; 271:30709-16.
4. Doyle H A, Mamula M J. Posttranslational modifications of selfantigens. Ann N Y Acad Sci 2005; 1050:1-9.
5. Rogers G E. Occurrence of citrulline in proteins. Nature 1962; 194: 1149-51.
6. Rogers G E, Simmonds D H. Content of citrulline and other amino-acids in a protein of hair follicles. Nature 1958; 182:186-7.
7. Schellekens G A, de Jong B A, van den Hoogen F H, van de Putte L B, van Venrooij W J. Citrulline is an essential constituent of antigenic determinants recognized by rheumatoid arthritis-specific autoantibodies. J Clin Invest 1998; 101:273-81.
8. Klareskog L, Ronnelid J, Lundberg K, Padyukov L, Alfredsson L. Immunity to citrullinated proteins in rheumatoid arthritis Annu Rev Immunol 2008; 26:651-75.
9. Hagiwara T, Nakashima K, Hirano H, Senshu T, Yamada M. Deimination of arginine residues in nucleophosmin/B23 and histones in HL-60 granulocytes. Biochem Biophys Res Commun 2002; 290:979-83.
10. Wang Y, Wysocka J, Sayegh J, Lee Y H, Perlin J R, Leonelli L, et al. Human PAD-4 regulates histone arginine methylation levels via demethylimination. Science 2004; 306:279-83.
11. Cuthbert G L, Daujat S, Snowden A W, Erdjument-Bromage H, Hagiwara T, Yamada M, et al. Histone deimination antagonizes arginine methylation. Cell 2004; 118:545-53.
12. Thompson P R, Fast W. Histonc citrullination by protein argininc deiminase is arginine methylation a green light or a roadblock? ACS Chem Biol 2006; 1:433-41.
13. Shirai H, Blundell T L, Mizuguchi K. A novel superfamily of enzymes that catalyze the modification of guanidino groups. Trends Biochem Sci 2001; 26:465-8.
14. Vossenaar E R, Zendman A J, van Venrooij W J, Pruijn G J. PAD, a growing family of citrullinating enzymes: genes, features and involvement in disease. BioEssays 2003; 25:1106-18.
15. Nakashima K, Hagiwara T, Yamada M. Nuclear localization of peptidylarginine deiminase V and histone deimination in granulocytes. J Biol Chem 2002; 277:49562-8.
16. Arita K, Hashimoto H, Shimizu T, Nakashima K, Yamada M, Sato M. Structural basis for Ca2_-induced activation of human PAD-4. Nat Struct Mol Biol 2004; 11:777-83. AUTOCITRULLINATION OF PAD-4 REGULATES PROTEIN CITRULLINATION 1639
17. Kinloch A, Lundberg K, Wait R, Wegner N, Lim N H, Zendman A J, et al. Synovial fluid is a site of citrullination of autoantigens in inflammatory arthritis. Arthritis Rheum 2008; 58:2287-95.
18. Matsuo K, Xiang Y, Nakamura H, Masuko K, Yudoh K, Noyori K, et al. Identification of novel citrullinated autoantigens of synovium in rheumatoid arthritis using a proteomic approach. Arthritis Res Ther 2006; 8:R175.
19. Chang X, Yamada R, Suzuki A, Sawada T, Yoshino S, Tokuhiro S, et al. Localization of peptidylarginine deiminase 4 (PADI4) and citrullinated protein in synovial tissue of rheumatoid arthritis. Rheumatology (Oxford) 2005; 44:40-50.
20. Suzuki A, Yamada R, Chang X, Tokuhiro S, Sawada T, Suzuki M, et al. Functional haplotypes of PADI4, encoding citrullinating enzyme peptidylarginine deiminase 4, are associated with rheumatoid arthritis. Nat Genet. 2003; 34:395-402.
21. Kang C P, Lee H S, Ju H, Cho H, Kang C, Bae S C. A functional haplotype of the PADI4 gene associated with increased rheumatoid arthritis susceptibility in Koreans. Arthritis Rheum 2006; 54: 90-6.
22. Hoppe B, Haupl T, Gruber R, Kiesewetter H, Burmester G R, Salama A, et al. Detailed analysis of the variability of peptidylarginine deiminase type 4 in German patients with rheumatoid arthritis: a case-control study. Arthritis Res Ther 2006; 8:R34.
23. Plenge R M, Padyukov L, Remmers E F, Purcell S, Lee A T, Karlson E W, et al. Replication of putative candidate-gene associations with rheumatoid arthritis in _{—}4,000 samples from North America and Sweden: association of susceptibility with PTPN22, CTLA4, and PADI4. Am J Hum Genet 2005; 77:1044-60.
24. Harris M L, Darrah E, Lam G K, Bartlett S J, Giles J T, Grant A V, et al. Association of autoimmunity to peptidyl arginine deiminase type 4 with genotype and disease severity in rheumatoid arthritis. Arthritis Rheum 2008; 58:1958-67.
25. Takizawa Y, Sawada T, Suzuki A, Yamada R, Inoue T, Yamamoto K. Peptidylarginine deiminase 4 (PADI4) identified as a conformation-dependent autoantigen in rheumatoid arthritis. Scand J Rheumatol 2005; 34:212-5.
26. Arita K, Shimizu T, Hashimoto H, Hidaka Y, Yamada M, Sato M. Structural basis for histone N-terminal recognition by human peptidylarginine deiminase 4. Proc Natl Acad Sci USA 2006; 103: 5291-6.
27. Halvorsen E H, Pollmann S, Gilboe I M, van der Heijde D, Land-ewe R, Odegard S, et al. Serum IgG antibodies to peptidylarginine deiminase 4 in rheumatoid arthritis and associations with disease severity. Ann Rheum Dis 2008; 67:414-7.
28. Halvorsen E H, Haavardsholm E A, Pollmann S, Boonen A, van der Heijde D, Kvien T K, et al. Serum IgG antibodies to peptidylarginine deiminase 4 predict radiographic progression in patients with rheumatoid arthritis treated with tumour necrosis factor-_blocking agents. Ann Rheum Dis 2009; 68:249-52.

1. A peptidylarginine deiminase 4 (PAD-4) polypeptide comprising one or more citrullinated Arginine sites.

2. The PAD-4 polypeptide of claim 1, wherein the one or more citrullinated Arginine sites are selected from the group consisting of Arg-8, Arg-123, Arg-131, Arg-137, Arg-156 Arg-205, Arg-212, Arg-218, Arg-292, Arg-372, Arg-374, Arg-383, Arg-394, Arg-419, Arg-427, Arg-441, Arg-484, Arg-488, Arg-495, Arg-536, and Arg-544, Arg-550, Arg-555, Arg-609, Arg-639, Arg-650, and Arg-651.

3. The PAD-4 polypeptide of claim 1, wherein the one or more citrullinated Arginine sites are selected from the group consisting of Arg-205, Arg-212, Arg-218, Arg-372, Arg-374, Arg-383, Arg-394, Arg-495, Arg-536, and Arg-544.

4. The PAD-4 polypeptide of claim 1, wherein the citrullinated Arginine sites comprise Arg-205, Arg-212, Arg-218, Arg-372, Arg-374, Arg-383, Arg-394, Arg-495, Arg-536, and Arg-544.

5. The PAD-4 polypeptide of claim 1, wherein the citrullinated Arginine sites comprise Arg-372 and Arg-374.

6. A PAD-4 polypeptide comprising a citrulline residue at the following sites: Arg-205, Arg-212, Arg-218, Arg-372, Arg-374, Arg-383, Arg-394, Arg-495, Arg-536, and Arg-544.

7. A PAD-4 polypeptide comprising a citrulline residue at the following sites: Arg-372 and Arg-374.

8. A method for detecting the presence of autoantibodies to citrullinated PAD-4 (cit-PAD-4 autoantibodies) in a subject comprising:
a. contacting a biological sample taken from a subject with a polypeptide of claim 7; and
b. detecting the binding of the polypeptide with an autoantibody specific for the polypeptide, wherein the detection of binding is indicative of the presence of cit-PAD-4 autoantibodies in the subject.

9. The method of claim 8, wherein the binding is detected by enzyme-linked immunosorbent assay (ELISA), immunoprecipitation or immunoblotting.

10. A method for assessing efficacy of an RA treatment regimen in a subject comprising:
a. establishing a baseline level of cit-PAD-4 autoantibodies in a subject prior to an RA treatment regimen;
b. monitoring the level of cit-PAD-4 autoantibodies using a polypeptide of claim 7 at least at one point after initiation of the RA treatment regimen; and
c. comparing the observed level of cit-PAD-4 autoantibodies to the baseline level of cit-PAD-4 autoantibodies, wherein a decrease in the level of PAD autoantibodies is indicative of the efficacy of the RA treatment regimen.

11. The method of claim 10, wherein the level of cit-PAD-4 autoantibodies is measured by ELISA, immunoprecipitation or immunoblotting.

12. A method for qualifying RA status in a subject comprising
a. measuring the level of cit-PAD-4 autoantibodies in a biological sample from the subject; and
b. correlating the measurement with RA status.

13. The method of claim 12, wherein the level of cit-PAD-4 autoantibodies is measured using a polypeptide of claim 7.

14. The method of claim 12, wherein the level of cit-PAD-4 autoantibodies is measured by ELISA, immunoprecipitation or immunoblotting.

15. The method of claim 12, wherein RA status is selected from the group consisting of the risk of RA, the development of RA, the presence or absence of RA, the stage of RA, the subtype of RA, the prognosis for the subject, and the effectiveness of treatment of RA.

16. A method of inducing tolerance to citrullinated PAD-4 in a subject comprising administering to the subject a pharmaceutical composition comprise a citrullinated PAD-4 polypeptide (cit-PAD-4 polypeptide) in an amount effective to induce tolerance to citrullinated PAD-4 in the subject.

17. The method of claim 16, further comprising administering an immunosuppressive agent.

18. A vaccine comprising a cit-PAD-4 polypeptide, wherein the vaccine elicits an immune response that suppresses or eliminates cit-PAD-4 specific autoreactive T-cells in an individual.

19. A kit comprising a cit-PAD-4 polypeptide of claim 7.


SEQUENCE LISTING (14 sequences)

SEQ ID NO: 1 - DNA, 2265 bp, Homo sapiens
acagccagagggacgagctagcccgacgatggcccaggggacattgatccgtgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtac

SEQ ID NO: 2 - protein, 663 aa, Homo sapiens
MAQGTLIRVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 3 - protein, 663 aa, Artificial Sequence
  Citrullinated Sequence at all Arginine Sites.
MAQGTLIXVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITXTGKVKPTXAVKDQXTWTWGPCGQGAILLVNCDXDNLESSAMDCEDDEVLDSEDLQDMSLMTLSTKTPKDFFTNHTLVLHVAXSEMDKVXVFQATXGKLSSKCSVVLGPKWPSHYLMVPGGKHNMDFYVEALAFPDTDFPGLITLTISLLDTSNLELPEAVVFQDSVVFXVAPWIMTPNTQPPQEVYACSIFENEDFLKSVTTLAMKAKCKLTICPEEENMDDQWMQDEMEIGYIQAPHKTLPVVFDSPXNXGLKEFPIKXVMGPDFGYVTXGPQTGGISGLDSFGNLEVSPPVTVXGKEYPLGXILFGDSCYPSNDSXQMHQALQDFLSAQQVQAPVKLYSDWLSVGHVDEFLSFVPAPDXKGFXLLLASPXSCYKLFQEQQNEGHGEALLFEGIKKKKQQKIKNILSNKTLXEHNSFVEXCIDWNXELLKXELGLAESDIIDIPQLFKLKEFSKAEAFFPNMVNMLVLGKHLGIPKPFGPVINGXCCLEEKVCSLLEPLGLQCTFINDFFTYHIXHGEVHCGTNVXXKPFSFKWWNMVP

SEQ ID NO: 4 - protein, 663 aa, Artificial Sequence
  Citrullinated Sequence at Arg-205, Arg-212, Arg-218, Arg-372, Arg-374, Arg-383, Arg-394, Arg-495, Arg-536, and Arg-544.
MAQGTLIRVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVAXSEMDKVXVFQATXGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPXNXGLKEFPIKXVMGPDFGYVTXGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPXSCYKLFQEQQNEGHGEALLFEGIKKKKQQKIKNILSNKTLXEHNSFVEXCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 5 - protein, 663 aa, Artificial Sequence
  Citrullinated Sequence at Arg-372 and Arg-374.
MAQGTLIRVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPXNXGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 6 - protein, 11 aa, Artificial Sequence
  cit-PAD4 polypeptide sequence
VMGPDFGYVTX

SEQ ID NO: 7 - protein, 11 aa, Artificial Sequence
  cit-PAD4 polypeptide sequence
LLLASPXSCYK

SEQ ID NO: 8 - protein, 12 aa, Artificial Sequence
  cit-PAD4 polypeptide sequence
XVMGPDFGYVTR

SEQ ID NO: 9 - protein, 11 aa, Artificial Sequence
  cit-PAD4 polypeptide sequence
TLXEHNSFVER

SEQ ID NO: 10 - protein, 19 aa, Artificial Sequence
  cit-PAD-4 polypeptide sequence
DFFTNHTLVLHVAXSEMDK

SEQ ID NO: 11 - protein, 12 aa, Artificial Sequence
  cit-PAD-4 polypeptide sequence
XVMGPDFGYVTR

SEQ ID NO: 12 - protein, 14 aa, Artificial Sequence
  cit-PAD-4 polypeptide sequence
TLPWFDSPXNXGLK

SEQ ID NO: 13 - protein, 8 aa, Artificial Sequence
  cit-PAD-4 polypeptide sequence
VFQATXGK

SEQ ID NO: 14 - protein, 10 aa, Artificial Sequence
  cit-PAD-4 polypeptide sequence
VXVFQATXGK